SEQUENCE LISTING (5 sequences)

SEQ ID NO: 1 - DNA, 705 bp, Artificial Sequence
  Porcine circovirus-2 strain PCV2 capsid
atgacgtatccaaggaggcgtttccgcagacgaagacaccgcccccgcagccatcttggccagatcctccgccgccgcccctggctcgtccacccccgccaccgttaccgctggagaaggaaaaatggcatcttcaacacccgcctctcccgcaccatcggttatactgtcaagaaaaccacagtcagaacgccctcctggaatgtggacatgatgagatttaatattaatgattttcttcccccaggagggggctcaaaccccctcactgtgccctttgaatactacagaataaggaaggttaaggttgaattctggccctgctccccaatcacccagggtgacaggggagtgggctccactgctgttattctagatgataactttgtaacaaaggccaatgccctaacctatgacccctatgtaaactactcctcccgccataccataacccagcccttctcctaccactcccggtactttaccccgaaacctgtccttgataggacaatcgattacttccaacccaataacaaaagaaatcaactctggctgagactacaaactactggaaatgtagaccatgtaggcctcggcactgcgttcgaaaacagtatctacgaccaggactacaatatccgtataaccatgtatgtacaattcagagaatttaatcttaaagaccccccacttaaccctaagtga

SEQ ID NO: 2 - protein, 234 aa, Artificial Sequence
  Porcine circovirus-2 strain PCV2 capsid
MTYPRRRFRRRRHRPRSHLGQILRRRPWLVHPRHRYRWRRKNGIFNTRLSRTIGYTVKKTTVRTPSWNVDMMRFNINDFLPPGGGSNPLTVPFEYYRIRKVKVEFWPCSPITQGDRGVGSTAVILDDNFVTKANALTYDPYVNYSSRHTITQPFSYHSRYFTPKPVLDRTIDYFQPNNKRNQLWLRLQTTGNVDHVGLGTAFENSIYDQDYNIRITMYVQFREFNLKDPPLNPK

SEQ ID NO: 3 - DNA, 750 bp, Artificial Sequence
  Modified Porcine circovirus-2 (PCV2) capsid
atggagaaaatagtgcttctttttgcaatagtcagtcttgttaaaagtacgtatccaaggaggcgtttccgcagacgaagacaccgcccccgcagccatcttggccagatcctccgccgccgcccctggctcgtccacccccgccaccgttaccgctggagaaggaaaaatggcatcttcaacacccgcctctcccgcaccatcggttatactgtcaagaaaaccacagtcagaacgccctcctggaatgtggacatgatgagatttaatattaatgattttcttcccccaggagggggctcaaaccccctcactgtgccctttgaatactacagaataaggaaggttaaggttgaattctggccctgctccccaatcacccagggtgacaggggagtgggctccactgctgttattctagatgataactttgtaacaaaggccaatgccctaacctatgacccctatgtaaactactcctcccgccataccataacccagcccttctcctaccactcccggtactttaccccgaaacctgtccttgataggacaatcgattacttccaacccaataacaaaagaaatcaactctggctgagactacaaactactggaaatgtagaccatgtaggcctcggcactgcgttcgaaaacagtatctacgaccaggactacaatatccgtataaccatgtatgtacaattcagagaatttaatcttaaagaccccccacttaaccctaagtga

SEQ ID NO: 4 - protein, 249 aa, Artificial Sequence
  Modified porcine circovirus-2 strain PCV2 capsid
MEKIVLLFAIVSLVKSTYPRRRFRRRRHRPRSHLGQILRRRPWLVHPRHRYRWRRKNGIFNTRLSRTIGYTVKKTTVRTPSWNVDMMRFNINDFLPPGGGSNPLTVPFEYYRIRKVKVEFWPCSPITQGDRGVGSTAVILDDNFVTKANALTYDPYVNYSSRHTITQPFSYHSRYFTPKPVLDRTIDYFQPNNKRNQLWLRLQTTGNVDHVGLGTAFENSIYDQDYNIRITMYVQFREFNLKDPPLNPK

SEQ ID NO: 5 - DNA, 6778 bp, Artificial Sequence
  Pcdna3.4-PCV2. (Fig. 1A).
atgacttaccctagacgacggttccgaagacgcagacacagaccgcgatcacatctcggacagatccttcgaagaagaccttggctcgttcatccccggcacagatatagatggcgaagaaaaaatggaattttcaatacccgcctgagccgcactatcgggtacaccgtgaaaaagacgacagtgcgcaccccttcctggaatgtcgacatgatgcgcttcaacataaacgatttcctgccacccggaggaggaagtaatcccctgactgttcctttcgaatactatagaataagaaaagtgaaagtggaattctggccctgcagccccattacacagggagacagaggtgtaggctccaccgctgtgattcttgatgacaattttgtgacgaaagctaatgcactgacctacgacccctacgtgaattactctagcagacatacaattacccagcccttttcctaccattcccgatattttactccaaaacccgtccttgacagaactattgactattttcagcccaataataaacgcaaccaactctggctcagacttcagactactggcaacgtggatcacgtcggacttgggacagcgttcgagaactctatatacgaccaagactataacattcgcattacaatgtacgtgcagttcagagaattcaatctcaaagaccccccactcaatccaaagtgagcggccgccccgggttcgaaaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttaagcttggatccactagtgaattcatctaaggtaccagtccagctagcgccgccacc

(19) United States
(12) Patent Application Publication
(10) Pub. No.: US 2022/0177855 A1
(43) Pub. Date: Jun. 9, 2022

(54) RECOMBINANT CIRCOVIRUS CAPSID-VIRUS-LIKE PARTICLE (VLP): COMPOSITIONS, METHODS AND USES

(71) Applicants: TechnoVax, Inc., Elmsford, NY (US); HUVEPHARMA, Sofia (BG)

(72) Inventors: Jose M. GALARZA, Elmsford, NY (US); Ke WEN, Elmsford, NY (US); Boris GAVRILOV, Sofia (BG)

(21) Appl. No.: 17/602,923

(22) PCT Filed: Apr. 23, 2020
(86) PCT No.: PCT/US2020/029561

§ 371 Date: Oct. 11, 2021

Related U.S. Application Data
(60) Provisional application No. 62/837,758, filed on Apr. 24, 2019.

(51) Int. Cl. C12N 7/00; C12N 15/85; C07K 14/005

(52) U.S. Cl. CPC C12N 7/00; C12N 15/85; C12N 2750/10052; C12N 2750/10023; C07K 14/005

(57) ABSTRACT

Provided herein is a mammalian expression system for producing recombinant porcine circovirus type 2 (PCV2) virus-like particles (VLPs). The expression system includes a mammalian cell and a plasmid that comprises a PCV2 gene encoding a capsid protein. The PCV2 gene is codon optimized, and the mammalian cell is transfected with the plasmid. The expression system produces recombinant PCV2 VLPs, such as PCV2d VLPs. Also provided herein are a method for producing porcine circovirus type 2 (PCV2) virus-like particles (VLPs), as well as a PCV2 VLP generated by the method.

### CROSS REFERENCE TO RELATED PATENT APPLICATION

This application claims priority to and the benefit thereof from U.S. Patent Application No. 62/837,758, titled “Recombinant Circovirus Capsid-Virus-Like Particle (VLP): Compositions, Methods and Uses”, the entirety of which is hereby incorporated by reference herein.

### 1. FIELD

The present application relates to compositions comprising Porcine circovirus-2 (PCV2), and in particular PCV2 virus-like particles (VLPs).

### 2. BACKGROUND

Circoviruses are small nonenveloped icosahedral viruses that contain a small circular, covalently closed, single stranded DNA (ssDNA) genome. Circoviruses have been identified in a variety of species and are known to cause infections in avian, aquatic, and terrestrial animals (1, 2). The variation of the capsid morphology and genome organization has recently led to the classification of the Circoviridae family into two separate genera, Circovirus and Cyclovirus (3). The genus circovirus comprises porcine circoviruses types 1 (PCV1), 2 (PCV2), and 3 (PCV3) (2). Genome sequences associated with the Cyclovirus genus has been associated with several vertebrate and invertebrate species, although the recognition of definitive hosts for this group is still unclear (2). PCV2 infections are responsible for significant mortality among swine as the causative agent of porcine circovirus associated disease (PCVAD), and have also been associated with porcine dermatitis nephropathy syndrome (PDNS) as well as porcine reproductive disorders (1, 4).

The virion particle is approximately 19 nm in diameter, and the genome of PCV ranges from 1.7 kb to 2.3 kb in size. The circular nature of the genome has led to the viral family name circovirus. The evolutionary history has been explored and has allowed detailed phylogenetic trees and variations in the capsid surface structure to be described (5-8). The initial cryo-EM image reconstruction of several native circoviruses demonstrated that the capsid has T=1 icosahedral symmetry (9). The PCV genome encodes for one structural capsid protein (CP). Expression and purification of the PCV2b CP protein from E. coli were demonstrated to self-assemble and mimic the overall morphology of the 70 infectious viruses. The crystal structure of this virus-like particle (VLP) visualized the CP fold to be that of the canonical viral jelly roll consisting of two four stranded β-sheets (10, 11). The loops connecting the β-strands form the features on the viral surface and may include the antigenic epitopes. The PCV2b CP was also expressed and purified as VLP from Trichoplasia ni insect cells (11). The cryo-EM image reconstruction of this VLP demonstrated that the N-terminus is located inside the capsid, and the authors concluded that the antigenic properties associated with the N-terminus is likely a result of the N-terminus transiently externalizing from the capsid via a process referred to as viral “breathing” (11-13). The externalization of the N-terminus may play an important role in the life cycle of the virus.

The amino acid sequences of PCV2 entries in GenBank have been categorized into four different genotypes (PCVa-d) (14). PCV2a was the dominant global genotype until 2003 when a genotype shift to PCV2b was observed (15, 16). The PCV2c genotype may have become extinct as there are only three depositions in GenBank. In 2013 Wei et al. reported and deposited a large quantity of PCV2d sequences into GenBank (5). Additional reports and deposition of PCV2d sequences have resulted in 84 approximately 320 depositions in GenBank (7). The increase in the deposition of PCV2d sequences may be a result of PCV2d becoming an emerging and predominant genotype in Asia, Europe, North and South America. The increase may be a result of escape mutants of vaccination (17). Consequently, PCV2d may represent a second global genotype shift that may be unresponsive to the vaccination program currently in place for PCV2 (7). Despite the significant number of phylogenetic studies of the PCV2 genotypes and the emerging importance of PCV2d on the global swine industry there are no reports describing the structure of the PCV2d capsid.

Given the emergence of PCV2d, a greater understanding of the PCV2 genotypes and their differences are needed for the development of treatment for illnesses caused by these types of viruses. These and other concerns are addressed by the present application.

### 3. SUMMARY

In a first aspect, a mammalian expression system for producing recombinant porcine circovirus type 2 (PCV2) virus-like particles (VLPs) is provided. The expression system comprises a mammalian cell, and a plasmid comprising a PCV2 gene encoding a capsid protein. The PCV2 gene is codon optimized and the mammalian cell is transfected with the plasmid. The expression system produces recombinant PCV2 VLPs.

In another aspect, the mammalian cell is a human embryonic kidney-293 (HEK-293) mammalian cell.

In another aspect, the PCV2 gene includes a recognition site for NheI, a Kozak sequence, and a recognition site for NotI. The recognition site for NheI and the Kozak sequence are upstream from a start codon of the PCV2 gene, and the recognition site for NotI is incorporated after a termination codon of the PCV2 gene.

In another aspect, a majority of the produced recombinant PCV2 VLPs are present in the nucleus of the mammalian cells.

In another aspect, the capsid protein comprises an amino acid sequence of SEQ ID NO: 2. In another aspect, the capsid protein is encoded by a nucleotide sequence of SEQ ID NO: 1.

In another aspect, capsid protein is modified with a secretion signal sequence introduced at an NH2 terminal of the capsid protein. In a further aspect, the capsid protein comprises an amino acid sequence of SEQ ID NO: 4. In a further aspect, the capsid protein is encoded by a nucleotide sequence of SEQ ID NO: 3.

In another aspect, the produced recombinant PCV2 VLPs are selected from the group consisting of: PCV2a VLPs, PCV2b VLPs, PCV2c VLPs, PCV2d VLPs, and PCV2e VLPs. In a further aspect, the produced recombinant PCV2 VLPs are PVC2d VLPs.

In another aspect, the plasmid is pcDNA3.4-PCV2. In another aspect, the PCV2 gene is codon optimized using a codon-optimized amino acid sequence of FIG. 1A.

In a second aspect, a method for producing porcine circovirus type 2 (PCV2) virus-like particles (VLPs) is provided. In the method, a suspension of cultured mammalian cells is provided. The mammalian cells are transfected with a plasmid comprising a PCV2 gene encoding a capsid protein. Valproic acid (VPA) sodium salt is added to the transfected mammalian cells, and the addition of the VPA sodium salt inhibits cell proliferation. The transfected mammalian cells are centrifuged and washed. The centrifuged mammalian cells are then suspended in a phosphate buffered saline (PBS) solution. Multiple freeze and thaw cycles are performed on the mammalian cells, and then the mammalian cells are sonicated in multiple cycles. Two successive centrifugation cycles of the mammalians cells are then performed to produce the PCV2 VLPs, and a majority of the produced PCV2 VLPs are present in the nucleus of the mammalian cells.

In another aspect of the method, the step of centrifuging and washing the transfected mammalian cells comprises: centrifuging the mammalian cells at 2,000×g for 15 min, washing the mammalian cells with PBS solution, and centrifuging the mammalian cells again at 2,000×g for 15 min.

In another aspect of the method, the centrifuged mammalian cells are frozen at approximately −80° C. and thawed at approximately 37° C. during the freeze and thaw cycles.

In another aspect of the method, a first of the two successive centrifugation cycles is performed at 2,000×g for 15 min and a second of the two successive centrifugation cycles is performed at 8,000×g for 15 min.

In another aspect of the method, the PCV2 VLPs are purified by ultracentrifugation.

In another aspect of the method, the mammalian cells are human embryonic kidney-293 (HEK-293) mammalian cells.

In another aspect of the method, the PCV2 gene includes a recognition site for NheI, a Kozak sequence, and a recognition site for NotI. The recognition site for NheI and the Kozak sequence are upstream from a start codon of the PCV2 gene, and the recognition site for NotI is incorporated after a termination codon of the PCV2 gene.

In another aspect of the method, the plasmid is pcDNA3.4-PCV2.

In another aspect of the method, the produced PCV2 VLPs are selected from the group consisting of: PCV2a VLPs, PCV2b VLPs, PCV2c VLPs, PCV2d VLPs, and PCV2e VLP. In a further aspect, the produced PCV2 VLPs are PVC2d VLPs.

In a third aspect, a PCV2 VLP generated by above method is provided.

### 4. BRIEF DESCRIPTION OF DRAWINGS

FIGS. 1A-1D. Expression of PCV2 virus-like particles in mammalian cells. FIG. 1A) Plasmid generated for 404 expression of PCV2d capsid protein. The codon optimized PCV2d capsid gene was synthesized (Blue Heron Technologies, Bothell, Wash.) and cloned into expression vector pcDNA3.4 (Fisher Scientific). FIG. 1B) Protein expression was conducted in transiently transfected suspension cultures of Expi293 cells (Life Technologies). SDS-PAGE analysis of purified PCV2d VLPs (1 μg protein) and stained with Coomassie blue. FIG. 1C) SDS-PAGE analysis of purified PCV2d VLPs (0.5 μg protein) transferred to a nitrocellulose membrane and probed for a Western Blot with primary rabbit anti PCV2 capsid polyclonal antibody (Cab 183908, Abcam, UK). FIG. 1D) Negative stained electron microscopy micrograph of purified VLP stained with uranyl acetate. Particle sizes are approximately 19 nm diameter.

FIGS. 2A-2C. Structural study of the PCV2d VLP. FIG. 2A) Icosahedral cryo-EM image reconstruction of the purified PCV2d VLP colored according to the local resolution. The gradient color map on the left-hand side indicates the resolution for the colors. FIG. 2B) Ribbon cartoon of the atomic coordinates with one asymmetric unit colored in cyan. FIG. 2C) Structural overlay of the PCV2a (dark blue), PCV2b (cyan), and PCV2d (yellow). The loops are labeled according to the β-strands they connect. Figures generated using UCSF ChimeraX (47).

FIGS. 3A-3D. Sequence comparison of 1,377 PCV2 capsid protein entries plotted on the PCV2d atomic coordinates. FIG. 3A) The sequence alignment by the Clustal Omega server was used to generate the WebLogo diagram to demonstrate sequence variation. The horizontal axis of the alignment indicates the amino acid and the vertical axis indicates its observed frequency. Bars connecting amino acids 77, 80, 99, 91, 190, 19 (black), and 53, 215 (grey) represent the evolutionary coupled clusters shown in panel D. FIG. 3B) Space filling model of the PCV2d atomic coordinates with a modified color-coding scheme of ConSurf. The color bar at the bottom indicates the degree of conservation determined by the ConSurf server. The yellow box indicates insufficient data as determined by the server (1 indicates poorly conserved and 9 indicates highly conserved mutations). The top right quadrant of the VLP surface has been removed to display the sequence conservation in the interior of the capsid. Image made with UCSF ChimeraX and colored using flat lighting. FIG. 3C) Highly conserved amino acid patches on the capsid surface (amino acids 82,170,188,189 and 193 in green, 55, 56, 51 and 73 in blue). Antibodies directed against these residues may possess broadly neutralizing capability. FIG. 3D) Ribbon cartoon of a PCV2d subunit. Residues in sick are evolutionary coupled together, as determined using the plmc. MATLAB 2019, and EVzoom programs. Figures generated using UCSF ChimeraX (47).

FIGS. 4A-4H. Sites of antibody neutralization. Top) Space filling model of the PCV2d atomic coordinates with the surface exposed amino acids colored in cyan, sequence variable amino acids in purple, and antibody binding amino acids in yellow. Middle) WebLogo diagram of 11 amino acids containing the solvent exposed amino acids. Bottom) Amino acids on the surface of the capsid.

FIG. 5. Body weight evolution during the Study phase for each treatment group: T01 (PIGONE 1 mL), T02 (PIGONE 2 mL), T03_A (Huve-PCV2_A), T03_B (Huve-PCV2_B), T04 (HVP-DNA), T05 (CIRCOFLEX [Inactivated baculo-expressed PCV2 ORf2]) and T06 (PBS). The Y-axis represent the body weight in Kg and the X-axis the Study days from SD0 to SD63.

FIG. 6. Average daily weight gain during the Study periods for each treatment group: T01 (PIGONE 1 mL), T02 (PIGONE 2 mL), T03_A (Huve-PCV2_A), T03_B (Huve-PCV2_B), T04 (HVP-DNA), T05 (CIRCOFLEX (Inactivated baculo-expressed PCV2 ORf2)) and T06 (PBS). The Y-axis represent the weight gain in gr and the X-axis the Study periods SD0-SD35; SD35-SD63; SD0-SD63.

FIG. 7. Serology evolution during the Study phase for each treatment group: T01 (PIGONE 1 mL), T02 (PIGONE 2 mL), T03_A (Huve-PCV2_A), T03_B (Huve-PCV2_B), T04 (HVP-DNA), T05 (CIRCOFLEX [Inactivated baculo-expressed PCV2 ORf2]) and T06 (PBS). The Y-axis represent the S/P value and the X-axis the Study days from SD0 to SD63.

FIG. 8. Percentage of quantifiable positive and non-quantifiable positive animals during the challenge phase for each treatment group: T01 (PIGONE 1 mL), T02 (PIGONE 2 mL), T03_A (Huve-PCV2_A), T03_B (Huve-PCV2_B), T04 (HVP-DNA), T05 (CIRCOFLEX (Inactivated baculo-expressed PCV2 ORf2)) and T06 (PBS). The Y-axis represent the percentage of animals and the X-axis the Study days from SD35 to SD63.

FIG. 9. Mean viral loads of quantifiable positive animals (>4 log₁₀ copies/mL) during the challenge phase for each treatment group: T01 (PIGONE 1 mL), T02 (PIGONE 2 mL), T03_A (Huve-PCV2_A), T03_B (Huve-PCV2_B), T04 (HVP-DNA), T05 (CIRCOFLEX (Inactivated baculo-expressed PCV2 ORf2)) and T06 (PBS). The Y-axis represent the viral load and the X-axis the Study days from SD41 to SD63.

FIG. 10. Percentage of quantifiable positive, non-quantifiable positive and below limit of detection animals during the challenge phase for each treatment group: T01 (PIGONE 1 mL), T02 (PIGONE 2 mL), T03_A (Huve-PCV2_A), T03_B (Huve-PCV2_B), T04 (HVP-DNA), T05 (CIRCOFLEX (Inactivated baculo-expressed PCV2 ORf2)) and T06 (PBS). The Y-axis represent the percentage of animals and the X-axis the Study days from SD35 to SD63.

FIG. 11. Mean viral loads of all animals (>2 log₁₀ copies/mL) during the challenge phase for each treatment group: T01 (PIGONE 1 mL), T02 (PIGONE 2 mL), T03_A (Huve-PCV2_A), T03_B (Huve-PCV2_B), T04 (HVP-DNA), T05 (CIRCOFLEX (Inactivated baculo-expressed PCV2 ORf2)) and T06 (PBS). The Y-axis represent the viral load and the X-axis the Study days from SD35 to SD63.

### 5. DETAILED DESCRIPTION

The practice of the various embodiments of the present application can employ, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); Fundamental Virology, Second Edition (Fields & Knipe eds., 1991, Raven Press, New York).

All publications, patents and patent applications cited herein are hereby incorporated by reference in their respective entireties.

As used in this specification and the appended claims, the singular forms “a,” “an” and “the” include plural references unless the content clearly dictates otherwise. Thus, for example, reference to “a VLP” can include a mixture of two or more such VLPs.

### Definitions

As used herein, the “virus-like particle” or “VLP” refer to a nonreplicating, viral shell. VLPs are generally composed of one or more viral proteins, such as, but not limited to those proteins referred to as capsid, coat, shell, surface and/or envelope proteins, or particle-forming polypeptides derived from these proteins. VLPs can form spontaneously upon recombinant expression of the protein in an appropriate expression system. Methods for producing particular VLPs are discussed more fully below. The presence of VLPs following recombinant expression of viral proteins can be detected using conventional techniques known in the art, such as by electron microscopy, biophysical characterization, and the like. See, e.g., Baker et al., Biophys. J. (1991) 60:1445-1456; Hagensee et al., J. Virol. (1994) 68:4503-4505. For example, VLPs can be isolated by density gradient centrifugation and/or identified by characteristic density banding (e.g., Examples). Alternatively, cryoelectron microscopy can be performed on vitrified aqueous samples of the VLP preparation in question, and images recorded under appropriate exposure conditions. Additional methods of VLP purification include but are not limited to chromatographic techniques such as affinity, ion exchange, size exclusion, and reverse phase procedures.

As used herein, the term “hybrid” or “chimeric” refers to a molecule (e.g., protein or VLP) that contains portions thereof, from at least two different proteins. It will be apparent that a hybrid or chimeric molecule as described herein can include full-length proteins fused to additional heterologous polypeptides (full length or portions thereof) as well as portions proteins fused to additional heterologous polypeptides (full length or portions thereof). It will also be apparent that the hybrid or chimeric molecule can include wild-type sequences or mutant sequences in any one, some or all of the heterologous domains.

An “antigen” refers to a molecule containing one or more epitopes (i.e., “antigenic epitopes”), either linear, conformational or both, that will stimulate a host's immune-system to make a humoral and/or cellular antigen-specific response. In one or more embodiments, an epitope will include between about 7 and 15 amino acids, such as, 9, 10, 12 or 15 amino acids. The term includes polypeptides which include modifications, such as deletions, additions and substitutions (generally conservative in nature) as compared to a native sequence, so long as the protein maintains the ability to elicit an immunological response, as defined herein. These modifications may be deliberate, as through site-directed mutagenesis, or may be accidental, such as through mutations of hosts which produce the antigens.

An “immunogenic composition” is a composition that comprises an antigenic molecule where administration of the composition to a subject results in the development in the subject of a humoral and/or a cellular immune response to the antigenic molecule of interest.

“Purified” or “purification” general refers to isolation of a substance (compound, polynucleotide, protein, polypeptide, polypeptide composition) such that the substance comprises the majority percent of the sample in which it resides. Typically, in a sample a “purified” component comprises 50%, preferably 80%-85%, or more preferably 90-95% of the sample. Techniques for purifying polynucleotides and polypeptides of interest are well-known in the art and include, for example, ion-exchange chromatography, affinity chromatography and sedimentation according to density.

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence are determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence. Typical “control elements”, include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences, and/or sequence elements controlling an open chromatin structure see e.g., McCaughan et al. (1995) PNAS USA 92:5431-5435; Kochetov et al (1998) FEBS Letts. 440:351-355.

A “nucleic acid” molecule can include, but is not limited to, prokaryotic sequences, eukaryotic mRNA, cDNA from eukaryotic mRNA, genomic DNA sequences from eukaryotic (e.g., mammalian) DNA, and even synthetic DNA sequences. The term also captures sequences that include any of the known base analogs of DNA and RNA.

“Recombinant” as used herein to describe a nucleic acid molecule means a polynucleotide of genomic, cDNA, semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide. “Recombinant host cells,” “host cells,” “cells,” “cell lines,” “cell cultures,” and other such terms denoting prokaryotic microorganisms or eukaryotic cell lines cultured as unicellular entities, are used interchangeably, and refer to cells which can be, or have been, used as recipients for recombinant vectors or other transfer DNA, and include the progeny of the original cell which has been transfected. It is understood that the progeny of a single parental cell may not necessarily be completely identical in morphology or in genomic or total DNA complement to the original parent, due to accidental or deliberate mutation. Progeny of the parental cell which are sufficiently similar to the parent to be characterized by the relevant property, such as the presence of a nucleotide sequence encoding a desired peptide, are included in the progeny intended by this definition, and are covered by the above terms.

“Codon optimization” or “codon optimized” generally refers to gene engineering approaches that utilize synonymous codon changes in order to increase protein production.

Techniques for determining amino acid sequence “similarity” are well known in the art. In general, “similarity” means the exact amino acid to amino acid comparison of two or more polypeptides at the appropriate place, where amino acids are identical or possess similar chemical and/or physical properties such as charge or hydrophobicity. A so-termed “percent similarity” then can be determined between the compared polypeptide sequences. Techniques for determining nucleic acid and amino acid sequence identity also are well known in the art and include determining the nucleotide sequence of the mRNA for that gene (usually via a cDNA intermediate) and determining the amino acid sequence encoded thereby, and comparing this to a second amino acid sequence. In general, “identity” refers to an exact nucleotide to nucleotide or amino acid to amino acid correspondence of two polynucleotides or polypeptide sequences, respectively.

Two or more polynucleotide sequences can be compared by determining their “percent identity.” Two or more amino acid sequences likewise can be compared by determining their “percent identity.” The percent identity of two sequences, whether nucleic acid or peptide sequences, is generally described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:482-489 (1981). This algorithm can be extended to use with peptide sequences using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). Suitable programs for calculating the percent identity or similarity between sequences are generally known in the art.

A “vector” is capable of transferring gene sequences to target cells (e.g., bacterial plasmid vectors, viral vectors, non-viral vectors, particulate carriers, and liposomes). Typically, “vector construct,” “expression vector,” and “gene transfer vector,” mean any nucleic acid construct capable of directing the expression of one or more sequences of interest in a host cell. Thus, the term includes cloning and expression vehicles, as well as viral vectors. The term is used interchangeable with the terms “nucleic acid expression vector” and “expression cassette.”

As used herein, “subject” generally refers to any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The present systems described herein are intended for use in any of the above vertebrate species, since the immune systems of all of these vertebrates operate similarly.

By “pharmaceutically acceptable” or “pharmacologically acceptable” is meant a material which is not biologically or otherwise undesirable, i.e., the material may be administered to an individual in a formulation or composition without causing any unacceptable biological effects or interacting in a deleterious manner with any of the components of the composition in which it is contained.

As used herein, “treatment” refers to any of (i) the prevention of infection or reinfection, as in a traditional vaccine, (ii) the reduction or elimination of symptoms, and (iii) the substantial or complete elimination of the pathogen in question. Treatment may be effected prophylactically (prior to infection) or therapeutically (following infection).

As used herein the term “adjuvant” refers to a compound that, when used in combination with a specific immunogen (e.g. a VLP) in a formulation, will augment or otherwise alter or modify the resultant immune response. Modification of the immune response includes intensification or broadening the specificity of either or both antibody and cellular immune responses. Modification of the immune response can also mean decreasing or suppressing certain antigen-specific immune responses.

As used herein an “effective dose” generally refers to that amount of VLPs of the one or more embodiments of the present application sufficient to induce immunity, to prevent and/or ameliorate an infection or to reduce at least one symptom of an infection and/or to enhance the efficacy of another dose of a VLP. An effective dose may refer to the amount of VLPs sufficient to delay or minimize the onset of an infection. An effective dose may also refer to the amount of VLPs that provides a therapeutic benefit in the treatment or management of an infection. Further, an effective dose is the amount with respect to VLPs of the one or more embodiments of the present application alone, or in combination with other therapies, that provides a therapeutic benefit in the treatment or management of an infection. An effective dose may also be the amount sufficient to enhance a subject's (e.g., a human's) own immune response against a subsequent exposure to an infectious agent. Levels of immunity can be monitored, e.g., by measuring amounts of neutralizing secretory and/or serum antibodies, e.g., by plaque neutralization, complement fixation, enzyme-linked immunosorbent, or microneutralization assay. In the case of a vaccine, an “effective dose” is one that prevents disease and/or reduces the severity of symptoms.

As used herein, the term “effective amount” refers to an amount of VLPs necessary or sufficient to realize a desired biologic effect. An effective amount of the composition would be the amount that achieves a selected result, and such an amount could be determined as a matter of routine experimentation by a person skilled in the art. For example, an effective amount for preventing, treating and/or ameliorating an infection could be that amount necessary to cause activation of the immune system, resulting in the development of an antigen specific immune response upon exposure to VLPs of the present application. The term is also synonymous with “sufficient amount.”

As used herein, the term “multivalent” refers to VLPs which have multiple antigenic proteins against multiple types or strains of infectious agents.

As used herein the term “protective immune response” or “protective response” refers to an immune response mediated by antibodies against an infectious agent, which is exhibited by a vertebrate (e.g., a human), that prevents or ameliorates an infection or reduces at least one symptom thereof. VLPs of the present application can stimulate the production of antibodies that, for example, neutralize infectious agents, blocks infectious agents from entering cells, blocks replication of said infectious agents, and/or protect host cells from infection and destruction. The term can also refer to an immune response that is mediated by T-lymphocytes and/or other white blood cells against an infectious agent, exhibited by a vertebrate (e.g., a human), that prevents or ameliorates influenza infection or reduces at least one symptom thereof.

As used herein, the term “vaccine” refers to a formulation which contains VLPs of the present application, which is in a form that is capable of being administered to a vertebrate and which induces a protective immune response sufficient to induce immunity to prevent and/or ameliorate an infection and/or to reduce at least one symptom of an infection and/or to enhance the efficacy of another dose of VLPs. Typically, the vaccine comprises a conventional saline or buffered aqueous solution medium in which the composition(s) of the present application is suspended or dissolved. In this form, the composition(s) of the present application can be used conveniently to prevent, ameliorate, or otherwise treat an infection. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses.

In one or more embodiments, the present application relates to compositions comprising viruses of the Circoviridae family [e.g., Porcine circovirus-2 (PCV2) its different genotypes and serotypes or other members of the family], virus-like particles (VLPs), and to methods of making and using these VLPs, including the creation and production of virus-like particle (VLP)-based vaccines (e.g., monovalent, polyvalent, single particle universal or polyvalent, single particle mosaic or modified chimeric compositions) as well as its use for therapeutic delivery [(e.g., small molecules, nucleic acids, antibodies, enzymes (nanocarriers, nanobodies)] diagnostic, immunomodulatory functions and therapeutic indications. In particular, the present disclosure includes strategies and methods used for the development of novel monovalent, multivalent or universal porcine circovirus vaccines that are able to protect swine against infection with one or more serotypes, clades or antigenic variants of the porcine circovirus genus. Also described herein are VLP production methods (e.g., secretion systems) that produce VLPs that display certain antigenic configurations or modifications. These VLPs feature conformational native or chimeric epitopes relevant for the generation of an enhanced neutralizing immune response to the porcine circovirus or other virial agents. Single particle monovalent, bivalent, multivalent, universal or chimeric (e.g., different serotypes and genotypes such as PCV2a, PCV2b, PCV2c, PCV2d and PCV2e) VLPs are assembled and used to formulate vaccine compositions, which allows for immunization and subsequent protection against one or more serotypes or antigenically distinct virus (e.g. Asian, European or North American serotypes, etc.) VLPs with native, modified or reengineered capsid monomers enables the linking/conjugation of different molecular entities to the external surface of the particle (small or large molecular entities) or the encapsidation of such molecular entities within the structure of the particle via disassemble and reassemble of the VLPs or alternative packaging methods.

Furthermore, VLPs are also used for the diagnosis of infection or for therapeutic indications. VLP vaccines can be produced in suspension culture of eukaryotic cells and retained in the cells or released into the culture medium. After purification, concentration, and formulation the vaccine can be administered by any suitable route, for example, via either mucosal or parenteral routes, and induce an immune response able to protect against any or all porcine circovirus serotypes, antigenic variants, etc. VLPs comprising therapeutics, immunomodulatory functions and diagnostic application are also provided.

These and other aspects of the present compositions and methods are described in further detail below with reference to the accompany drawing figures and examples, in which one or more illustrated embodiments and/or arrangements of the PCV2 VLPs are shown. The compositions and methods of the present application are not limited in any way to the illustrated embodiments and/or arrangements. It should be understood that the compositions and methods as shown in the accompanying figures are merely exemplary of the compositions and methods of the present application, which can be embodied in various forms as appreciated by one skilled in the art.

In accordance with one or more embodiments of the present application, a mammalian expression system for producing recombinant porcine circovirus type 2 (PCV2) virus-like particles (VLPs) is provided. The expression system comprises a mammalian cell, and a plasmid comprising a PCV2 gene encoding a capsid protein. The PCV2 gene is codon optimized and the mammalian cell is transfected with the plasmid. The expression system produces recombinant PCV2 VLPs. In at least one embodiment, the mammalian cell is a human embryonic kidney-293 (HEK-293) mammalian cell.

In one or more embodiments, the PCV2 gene of the expression system can include a recognition site for NheI, a Kozak sequence, and a recognition site for NotI. The recognition site for NheI and the Kozak sequence are upstream from a start codon of the PCV2 gene, and the recognition site for NotI is incorporated after a termination codon of the PCV2 gene.

In at least one embodiment, wherein a majority of the produced recombinant PCV2 VLPs are present in the nucleus of the mammalian cells.

In one or more embodiments, the capsid protein comprises an amino acid sequence of SEQ ID NO: 2. In at least one embodiment, the capsid protein is encoded by a nucleotide sequence of SEQ ID NO: 1.

In at least one embodiment, the capsid protein is modified with a secretion signal sequence introduced at an NH2 terminal of the capsid protein. In one or more further embodiments, the capsid protein comprises an amino acid sequence of SEQ ID NO: 4. In one or more further embodiments, the capsid protein is encoded by a nucleotide sequence of SEQ ID NO: 3.

In one or more embodiments, the produced recombinant PCV2 VLPs can be at least one of the following: PCV2a VLPs, PCV2b VLPs, PCV2c VLPs, PCV2d VLPs, and PCV2e VLPs. In a preferred embodiment, the produced recombinant PCV2 VLPs are PVC2d VLPs.

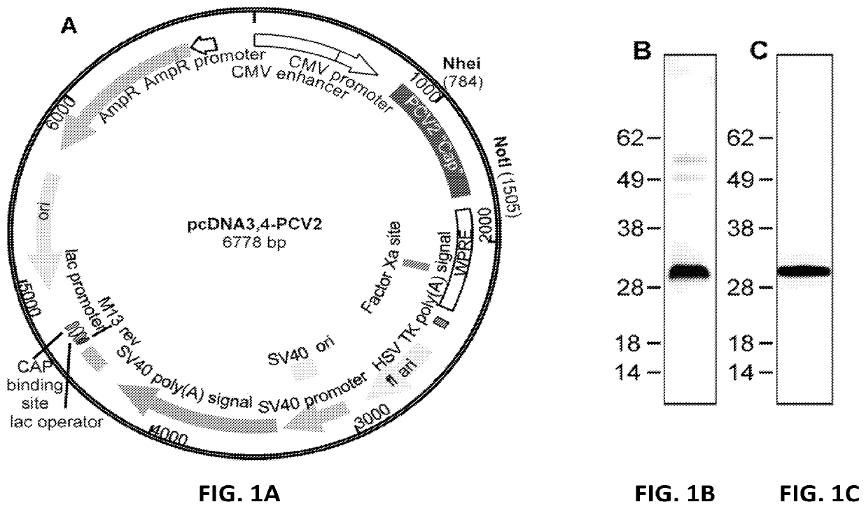

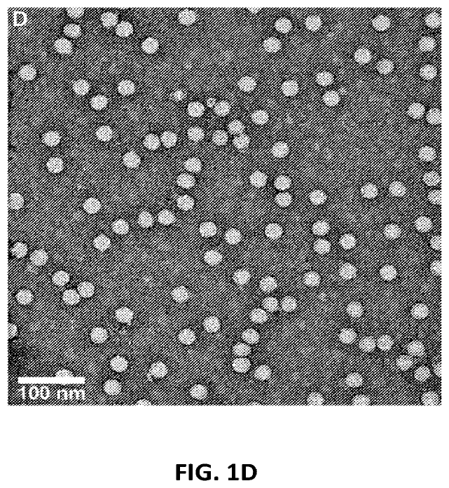

In at least one embodiment, the plasmid is pcDNA3.4-PCV2. In at one or more embodiments, the PCV2 gene is codon optimized using a codon-optimized amino acid sequence of FIG. 1A.

In one or more embodiments of the present application, a method for producing porcine circovirus type 2 (PCV2) virus-like particles (VLPs) is provided. In the method, a suspension of cultured mammalian cells is provided. The mammalian cells are transfected with a plasmid comprising a PCV2 gene encoding a capsid protein. Valproic acid (VPA) sodium salt is added to the transfected mammalian cells, and the addition of the VPA sodium salt inhibits cell proliferation. The transfected mammalian cells are centrifuged and washed. The centrifuged mammalian cells are then suspended in a phosphate buffered saline (PBS) solution. At least one freeze and thaw cycle, and preferably multiple freeze and thaw cycles, are performed on the mammalian cells, and then the mammalian cells are sonicated in multiple cycles. In one or more embodiments, two successive centrifugation cycles of the mammalians cells are then performed to produce the PCV2 VLPs. In at least one embodiment, a majority of the produced PCV2 VLPs are present in the nucleus of the mammalian cells.

In at least one embodiment, the step of centrifuging and washing the transfected mammalian cells can comprises: centrifuging the mammalian cells at 2,000×g for 15 min, washing the mammalian cells with PBS solution, and centrifuging the mammalian cells again at 2,000×g for 15 min.

In one or more embodiments, the centrifuged mammalian cells can be frozen at approximately −80° C. and thawed at approximately 37° C. during the freeze and thaw cycles.

In at least one embodiment, a first of the two successive centrifugation cycles is performed at 2,000×g for 15 min and a second of the two successive centrifugation cycles is performed at 8,000×g for 15 min.

In at least one embodiment, the PCV2 VLPs are purified by ultracentrifugation.

In at least one embodiment, the plasmid is pcDNA3.4-PCV2. In one or more embodiments of the method, the produced PCV2 VLPs are selected from the group consisting of: PCV2a VLPs, PCV2b VLPs, PCV2c VLPs, PCV2d VLPs, and PCV2e VLP. In at least one preferred embodiment, the produced PCV2 VLPs are PVC2d VLPs. In at least one embodiment, a PCV2 VLP generated by the above method is provided.

In summary, in accordance with one or more embodiments, the present application discloses a mammalian assembled system of PCV2 VLPs (e.g., PCV2d VLPs). The present application further discloses structural analyses based on cryo-electron microscopy reconstruction that reveal external and internal features of the VLPs, which have significant implications for the development of new PCV2 vaccines and utilization of the particles as nano-vehicles to deliver diverse molecular entities for prophylactic, therapeutic, immunotherapeutic, immunization (heterologous, homologous, multivalent), diagnostic amongst the various applications. These analyses and other examples related to PCV2 VLPs are explained in further detail below.

### 6. EXAMPLES

### 6.1. Example 1

Materials and Methods

Cells, Capsid Gene, Plasmid and antibody. Suspension cultures of Expi293F human cells (Life Technologies, CA) were grown in serum-free Expi293 expression medium (Life Technologies, CA) at 37° C. in a 5% CO2 environment and agitated at 150 rpm in Erlenmeyer flasks. The porcine circovirus type 2 (PCV2) gene encoding the capsid protein (strain RQ3) was chemically synthesized using a codon-optimized sequence (amino acid sequence shown in FIG. 1A) by Blue Heron Technologies (Bothell, Wash.). The recognition site for NheI and the Kozak sequence were added right upstream from the start codon, and the recognition site for NotI was incorporated after the termination codon. The synthesized Cap gene was recovered from the transport plasmid by a double digestion with NheI and 289 NotI restriction enzymes and sub-cloned after gel purification into the mammalian expression plasmid pcDNA3.4 cut with the same enzymes. The ligated plasmid was transformed into MAX Efficiency Stb12 Cells (Life Technologies) and a correct clone was identified via restriction enzyme analysis and verified by sequencing.

Viral-Like Particle (VLP) Production and Purification. PCV2 VLPs were produced in a suspension culture of Expi293F mammalian cells following transient transfection with the plasmid pcDNA3.4-PCV2 (FIGS. 1A-1D). Expi293F cells were seeded at the concentration of 2×10⁶ cells/ml and cultured for 16 h prior to transfection. Plasmid DNA (1 μg/ml) was diluted in a volume of Opti-MEM representing 5% of the total volume of the culture. Separately, polyethylenimine (PEI) was prepared in an equivalent volume of Opti-MEM (4 μg/ml). After 5 min of incubation at room temperature, the PEI solution was 300 added dropwise to the tube containing the DNA and after 30 min of incubation at RT the mixture was added to the cell suspension in a dropwise manner. Twenty-four hours after transfection, Valproic acid sodium salt (VPA) was added to the cell culture to a final concentration of 3.75 mM to inhibit cell proliferation. Seventy-two hours post-transfection the cells were pelleted by centrifugation at 2,000×g for 15 min, and then washed one time with phosphate buffered saline (PBS) and spun again at 2,000×g for 15 min. The cell pellet was re-suspended in PBS and then subjected to three freeze (−80° C.) and thaw (37° C.) cycles. Subsequently, the cells were further fragmented by three cycles of sonication and clarified by two successive centrifugations, first at 2,000×g for 15 min followed by 8,000×g for 15 min. This procedure released some of the VLP into the cytoplasm, but a majority of the particles appeared to be entrapped inside the cell nucleus (data not shown).

The PCV2 VLPs contained in the clarified supernatant were further purified by ultracentrifugation on a two-layer CsCl density gradient: lower layer, 5 ml of 1.4 g/ml CsCl and upper layer, 10 ml of 1.25 g/ml of CsCl both prepared in 10 mM Tris-HCl, (pH 7.9). Samples were loaded onto the gradient and spun at 15° C. for 4 h at 140,000×g using a SW28 rotor (Beckman Coulter, CA). The VLPs appeared as an opaque band at the interface of the 1.25 and 1.4 g/ml CsCl layers and were collected by piercing the tube with an 18G needle and syringe. The collected solution was mixed with 37% CsCl in 10 mM Tris-HCl (pH 7.9) to final volume of 12 ml and then spun at 15° C. for 16 hr at 155,000×g using a SW 41Ti rotor (Beckman Coulter, CA). The VLPs were detected at the lower part of the tube and recovered as described above. Collected VLP material was dialyzed against 10 mM Tris-HCl pH 7.9 and 150 mM NaCl at 10° C. overnight using a Slide-A-Lyzer Cassette. Purified PCV2 VLPs were concentrated and buffer exchanged to phosphate buffered saline (PBS) using Amicon Utra-4 centrifugal filter devices (Merck Millopore, MA). PCV2 VLP samples were stored in 50-100 μl aliquots at −80° C.

Western Blot and Coomassie Blue Stain. Purified VLPs were mixed with loading buffer, heated at 100° C. for 5 min and run on a 4 to 12% Bis-Tris SDS-polyacrylamide gel (Life Technologies, CA). Loading amounts of proteins were 1 μg for future Coomassie staining and 0.5 μg—for Western Blot. After electrophoretic separation, the gel was stained with Coomassie blue or proteins electro-transferred onto a 0.45 μm nitrocellulose membrane (Life Technologies LC2001). The membrane was then blocked with 5% non-fat milk in TBST (10 mM Tris-HCl, 130 mM NaCl, and 0.05% Tween-20, pH 7.4) for 1 h at (20° C.) followed by an overnight incubation at (20° C.) in primary Rabbit anti-porcine circovirus antibody (Cab 183908, Abcam, UK) diluted with blocking buffer. Membranes were washed 3 times with TBST and then incubated for 2 h with secondary antibody (goat anti-rabbit IgG HRP 332 conjugated, 1:1,000) diluted in blocking buffer. Finally, membranes were washed 3 times with TBST and developed with ECL Western blot system (Life Technologies, CA) according to manufacturer's instructions. The stained gel and immune blot images were acquired with a FluorChem Imager instrument (Protein Simple, CA).

337

Negative Staining and TEM Examination. 5 μL of pCV2-2 VLP samples was applied to CF200-CU carbon film 200 mesh copper grids (Electron Microscopy Science) for 1 min and the grids were then washed with 200 μl of 50 mM of Na Cacodylate buffer and then strained immediately with 50 μl of 0.5% uranyl acetate for 1 min. The grids were examined by a JOEL 2100 transmission electron microscope operating at 200 kV with an Orius 2048×2048 pixel CCD (Gatan Inc., Pleasanton, Calif.).

Cryo-EM Data Collection. Frozen hydrated samples of PCV2d VLPs were prepared on Quantifoil R 2/2, 200 mesh copper grids (Electron Microscopy Science). A 4 μl pre-screened sample of the VLP was applied to the grid blotted for 3 seconds and flash frozen in liquid ethane using a FEI Vitrobot instrument. The grids were stored in liquid nitrogen until data collection. Data was collected at cryogenic temperatures on a FEI Titan Krios, operating at 300 kV, with Gatan K2 camera post a GIF quantum energy filter with a width of 15 eV. Data collection was performed with the Leginon suite (36).

Image Reconstruction. The MotionCor2 package was used to correct for particle motion (37). Default parameters and dose weighting were used for the correction, with the exception of the patch 5 option, and the first frame of each movie was discarded during the alignment. The particles were selected automatically using Gautomatch v0.53, and contrast transfer function (CTF) estimation was performed on the aligned micrographs using Gctf v0.50 (38). Relion 3.0 was used to extract 23,358 300×300 pixel particles from the dose-weighted micrographs using the coordinates identified by Gautomatch. Reference free 2D classification was performed with Relion 3.0. Non-default options for this step included a diameter of 250 Å and 128 classes were requested (39). An initial model was generated to 60 Å resolution using the PCV2 crystal structure (PDB entry 3R0R) with the molmap function of UCSF Chimera (40). 3D classification was carried out on 7196 particles using Relion 3.0 with a diameter of 250 Å, 3 classes, and C1 symmetry. A single class with 4,442 particles exhibited the highest resolution. These particles were used for a high-resolution image reconstruction with Relion 3.0. Again, a diameter of 250 Å and I1 symmetry were used with the remaining default parameters of Relion 3.0. A binary mask was created using the relion_mask_create program of Relion 3.0. The binary mask for postprocessing was generated as follows: 1) the high resolution image reconstruction was low pass filtered to 15 Å resolution using relion_image_handler (Relion 3.0), 2) the lowest threshold at which noise exterior to the PCV2 capsid was identified for this volume using UCSF Chimera, 3) relion_mask_create (Relion 3.0) was used to convert this volume into a binary mask with the identified threshold, mask dilation by 7 pixels and 2 soft edge pixels. The resulting mask was then inspected with UCSF Chimera to ensure that no internal cavities existed.

Local resolution was calculated with the program MonoRes. The same binary mask used during the postprocessing with Relion was used for the calculation. A resolution range of 2.8 Å to 6.0 Å was used (41).

Structure refinement 376.

The atomic coordinates for the crystal structure of PCV2b crystal structure (PDB entry 3R0R) were modified using Coot (42). The biological matrices necessary to generate a virus-like particle are present in the PDB and were used by Coot to generate a VLP of PCV2d. UCSF Chimera was used to manually dock the VLP into the symmetrized image reconstructions. The resulting coordinates were iteratively refined using phenix.real_space_refine from the Phenix software package with non-crystallographic symmetry (NCS) constraints applied, and manual fitting with Coot (43).

Sequence Alignment and Evolution Coupling

A protein Blast search with the sequence of PCV2d and the Organism common name Porcine circovirus 2 (taxid:85708) filter generated a total of 1,966 PCV2 sequences (44). Partial sequences, sequences with names containing “putative”, “P3”, “unknown”, and “P27.9” were manually removed. Sequence alignment was performed on the remaining sequences using MUSCLE with default parameters (45). Sequences that generated gaps, possessed more than ten amino acids with a distinct sequence, or had no sequences similar to them were manually removed. This was done in order to remove spurious errors/artifacts that may have occurred during the sequencing process (i.e. artificial recombination during PCR with Taq polymerase (46)). Several rounds of alignment and deletion were performed to remove identical sequences. As a result, 1,377 sequences remained. The final round of alignment was performed with Clustal Omega with default parameters (19). Evolutionary coupling calculations could not be successfully performed with the EVcouplings server 394 (evfold.org) because of the limited range in the Expect (E) value present the sequence (i.e. the sequences are too similar). Consequently, plmc was used to generate coevolution and covariation within the sequences. The L2 lambda for fields and couplings used were 0.01 and 16.0, respectively, and a maximum number of 100 iterations were performed. The results were converted for visualization with EVzoom using MATLAB 2019 and the scripts provided by the plmc program (22). The resulting matrix was visualized with EVzoom (22), and structural covariance was visually confirmed with UCSF Chimera (40).

Results

Recombinant PCV2 VLPs assembled in mammalian cells. The PCV2d capsid protein (CP) gene (GenBank: ABX71783.1) was sub-cloned into the mammalian expression plasmid pcDNA 3.4 in order to produce recombinant PCV2d VLP in mammalian cells (FIG. 1A). Transfection of suspension culture of HEK-293 mammalian cells with the plasmid results in the production of the CP and assembly of the VLP. To date PCV2 VLP has been produced using insect cells, yeast, or E. coli (11), thus expression of VLP using mammalian cells provides an analogous substrate to that utilized by natural PCV2 infection. During the purification, it was determined that the majority of the VLP or CP was in the nucleus of the cell, while some was detected in the cytoplasm of the infected cell. This suggests that the VLP can exit the nucleus, possibly via a cryptic nuclear export signal that could not be confidently identified using bioinformatic tools (data not shown). Analysis of purified PCV2 VLP and its protein composition was confirmed by SDS-PAGE and Western blot analysis (FIGS. 1B and C). Further examination by negative stained electron microscopy showed homogeneous spherical particles with smooth edges, slightly rough surface with a diameter of ˜19 nm (FIG. 1D). The CP described in this study possesses the amino acid sequence identified from a number of recently isolated and reported PCV2d virus genome entries in GenBank, such as C/2013/3 isolated in Taiwan (AWD32058.1), W233-12 isolated in in Japan (BBE28610.1), CN-FJC011 isolated in China (AVZ66019.1), and England/15-P0222-09-14 isolated in England (AATN97185.1).

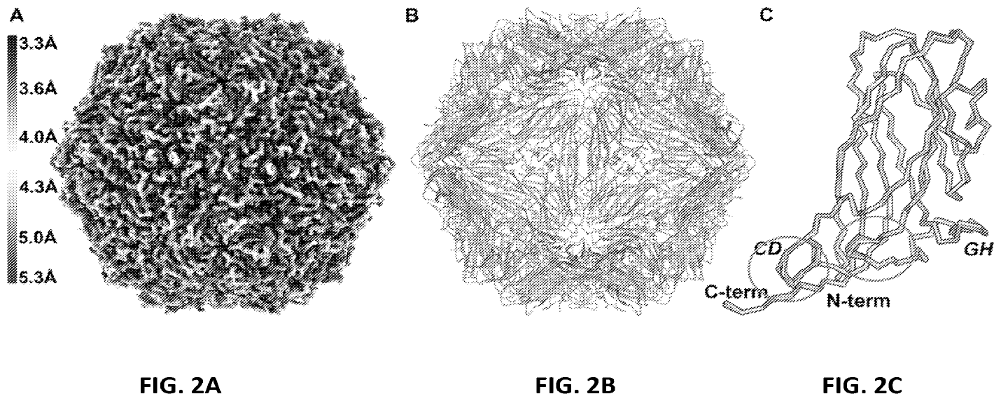

Cryo-electron microscopy and image reconstruction of PCV2d VLP. An icosahedral cryo-EM image reconstruction of purified PCV2d VLP was determined to a resolution of 3.3 Å (FIG. 2A). Close inspection of the image reconstruction indicates that the molecular envelopes of the side chains are of sufficient quality for modeling the atomic coordinates. The coordinates from the PCV2b crystal structure were manually fitted into the image reconstruction and appropriate modifications were performed to reflect the PCV2d amino acid sequence. The fitted model was refined through several iterations of automatic refinement with Phenix and manual adjustment using the program Coot (FIG. 2B). The final refinement statistics are shown in Table 1 below.

TABLE 1

| | Unliganded PCV2 | |
|---|---|---|
| Number of Micrographs | 799 | |
| Defocus range | −0.68 to −2.81 | μm |
| Total dose | 64 | e- Å⁻² |
| Dose rate | 6.4 | e⁻ Å⁻² sec⁻¹ |
| Number of Frames | 50 | |
| Particles extracted | 23,358 | |
| Particles used for reconstruction | 4,442 | |
| FSC resolution* | 3.3 | Å |
| B-factor sharpening ^{†} | −125.9 | Å² |
| CC | 87% | |
| B-factor average | 61.7 | Å² |

| RMSD ^{‡} | 0.008 |
|---|---|
| RMSD ^{§} | 0.783 |
| Molprobity ^{¶} | 1.84 |
| EMRinger | 5.3 |
| Ramachandran favored (%) | 95.2% |
| Ramachandran allowed (%) | 4.81% |
| EMDB | 6NLS |
| PDB | 9396 |

*Fourier shell correlation reported by Relion 3.0 using the gold standard method at a CC of 0.143.
^{†} The B-factor sharpening reported by Relion 3.0 during the post-refinement process.
^{‡} The root-mean standard deviation for bonds reported by Phenix.real_space_refine.
^{§} The root-mean standard deviation for angles reported by Phenix.real_space_refine.
^{¶} Molprobity Overall score.

Subunits from the PCV2a, b, and d genotype structure were superimposed and generated root-mean standard deviation plots of equivalent Ca atoms (9, 18). The regions that exhibit the greatest diversity correspond to two of the surface-exposed loops that consist of amino acids 85-91 (loop CD), and 188-194 (loop GH) (FIG. 2C).

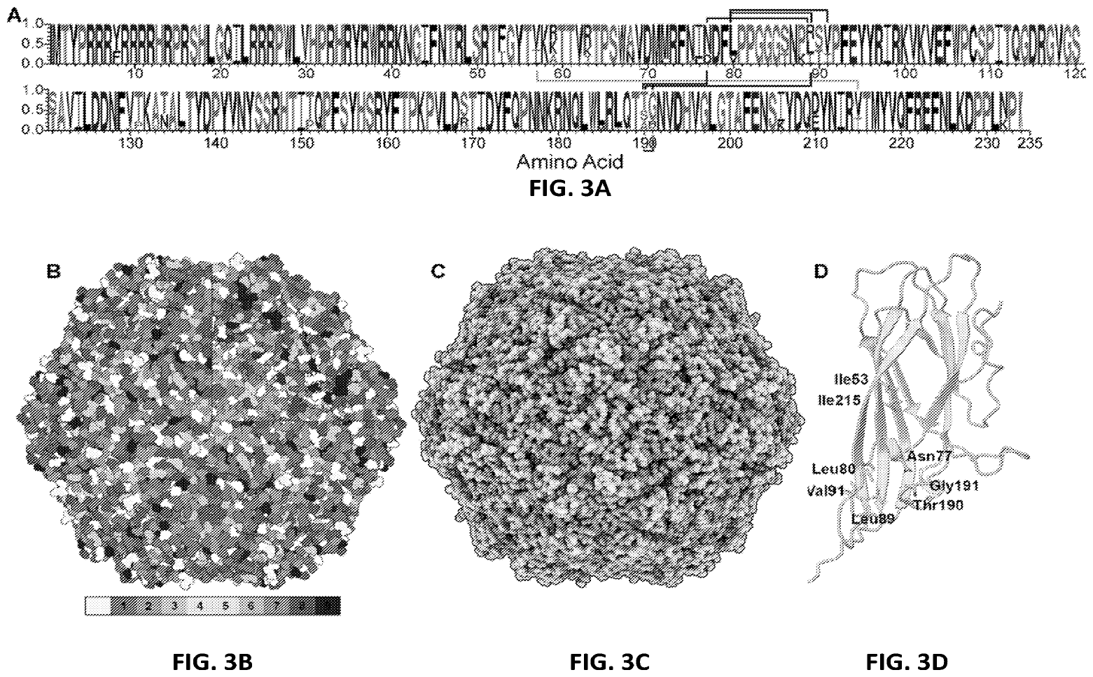

Sequence conservation among the four genotypes may help generate a universal vaccine for PCV2. There have been a number of publications where the amino acid sequence of the PCV2 CP are compared to attain insight into the evolution of the PCV2 capsid (6, 7). However, we are not aware of study where the sequence alignment information has been mapped onto the atomic coordinates of the capsid to understand how the observed mutations correlate with the structure and or immune evasion. A total of 1,377 non-redundant PCV2 CP sequences was subjected to sequence alignment by the Clustal Omega server (19). The sequence alignment was used to generate a WebLogo diagram to observe the sequence conservation (FIG. 3A). The horizontal axis of the alignment indicates the amino acid and the vertical axis indicates the observed frequency of the amino acid. The sequences with the greatest diversity (GenBank entries: AVZ65995.1 and ALK0432.1) share 74.4% sequence identity. While the WebLogo diagram successfully demonstrates the frequency of occurrence for the most popular amino acid(s) at each position, it dampens/eliminates the frequency of occurrence for the less popular amino acids(s). Indeed, mutations can be observed within the hydrophobic core of the protein, at the intersubunit interface, and residues on the outer and inner surface of the capsid. Moreover, closer inspection of the aligned sequences indicates that only the Met1 and Arg147 are absolutely conserved. The observed sequence diversity suggests that the capsid is capable of tolerating mutations at nearly every position while remaining an infectious virus. To visualize the sequence conservation/variability at each amino acid position, the sequence alignment information was plotted onto the PCV2d atomic coordinates using the ConSurf server (FIG. 3B) (20). The figure displays a significant degree of nonconserved mutations experienced by the capsid. Regions colored in red experience the greatest degree of change while regions in blue experience the least degree of change. Highly conserved residues are peppered across the structure; however, two sets of residues form patches on the surface of the capsid. These include Tyr55, Thr56, Met71 and Arg73, and Pro82, Thr170, Gln188, Thr189 and Val193 (FIG. 3C). The presence of these patches suggests that it may be possible to generate vaccines capable of neutralizing all available genotypes of PCV2 if antibody production could be directed at these amino acids (FIG. 3C).

Evolutionary coupled mutations differentiate the PCV2 genotypes. The large number of CP sequence deposition in GenBank allowed us to ask if any of the amino acid positions are evolutionary constrained (21). For evolutionary constrained amino acids, mutation of one amino acid requires mutation of the other amino acids. In the simplest of cases this may be because the amino acids pack against one another in the structure of the protein, such that mutation to a larger amino acid in one position requires mutation to a smaller amino acid in the second position for proper packing to occur. Such information can be used to predict the fold of a protein or identify functionally important sites (21-23). Evolutionary coupling (EC) measurements determined from the 1,377 unique sequences indicates that two independent locations in the structure demonstrate coupling. The first location is composed of amino acids 53 and 215, and the second location is composed of amino acids 77, 80, 89, 90, 190 and 191 (FIG. 3C). Surprisingly, sequence differences in the second location are responsible for the structural diversity observed between PCV2a and PCV2b/d for loops CD and GH (FIG. 2C) (9, 18). The EC results may indicate that the coupled residues are functionally relevant. Solvent accessible surface area calculations with the GetArea server (curie.utmb.edu) indicates that residues 77, 80, 89, and 90 are more than 75% buried, while residues 190 and 191 are exposed to the solvent (24). Residues 190 and 191 help define a neutralizing epitope on the surface of the capsid.

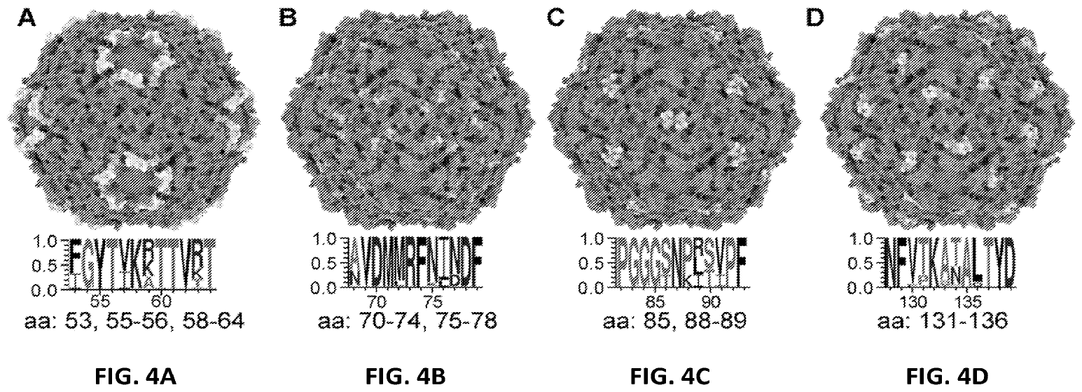

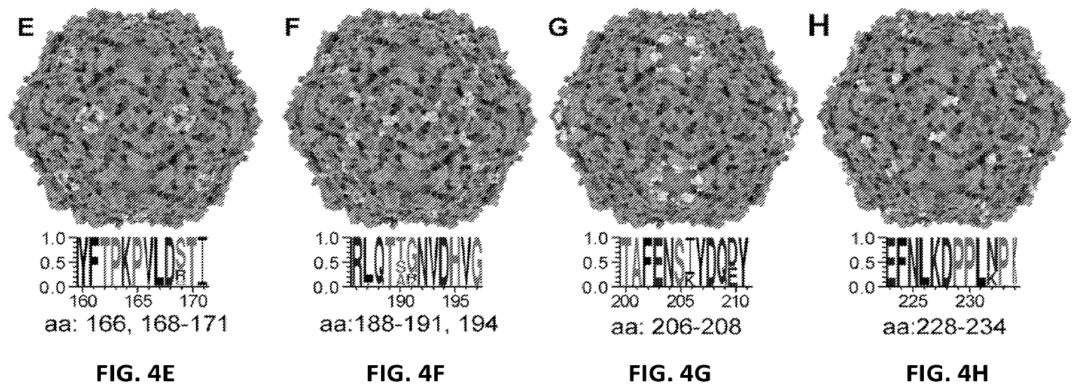

Sequence variation on the surface of the capsid is a response to neutralizing antibodies. The atomic coordinates of PCV2d were used to identify amino acids on the surface of the VLP with side chains exposed to solvent (53, 55-56, 58-64, 70-71, 73, 75, 77-78, 82-83, 85, 88-89, 102, 113, 123, 127, 131-137, 148, 155, 156, 158, 161, 166, 168-170, 188-191, 194, 204, 206-208, 210, 229-234). These amino acids may be antigenic determinants, as the side chains provide a surface for antibody interaction. Continuous sequences that may represent linear epitopes are shown in FIG. 4A-4H. The most variable amino acids in these regions were then identified (53, 57, 59, 60, 63, 75, 77, 88, 89, 131, 133, 134, 136, 169, 190, 191, 211, 215, and 232). The variation may be a result of escape mutants from antibody neutralization. The presence of mutations as an escape response to antibody neutralization is supported by antigenic subtyping experiments by Lefebvre et al. and Saha et al., where panels of neutralizing monoclonal antibodies are tested for their ability to bind to different strains of PCV2. The studies demonstrated that amino acid positions 59, 63, 88, 89, 130, 133, 206 and 210 are responsible for differentiating antibody binding (25, 26). A study by Franzo et. al. describing the evolution of PCV2 before and after the introduction of vaccination identified changes in amino acids 59, 191, 206, 210, 228 and 232 (27). Except for amino acid 130, which is buried in a subunit-subunit interface, these amino acids are on the surface of the capsid and undergo mutations (FIG. 4A-4H).

The inner region of the PCV2 VLP can be used to package material for nanotechnology. A near-central reconstruction slice was extracted from the cryo-EM image reconstruction and the density trace of pixel values was calculated in the horizontal and vertical directions. Based upon the central slice of the reconstruction, the PCV2 VLP outer diameter is approximately 18.5 nm assuming a roughly spherical shape for calculation. The outer volume is therefore estimated to be 3.3×10³ nm³. The inner diameter using the same spherical shape estimation has a diameter of approximately 13 nm. Therefore, the inner region volume is approximately 1.2×10³ nm³. Scans of a central slice of the 3D molecular volume demonstrated that the inner region is occupied. The density is lower than the capsid shell likely representing the cumulative disordered matrix N-terminus of the 60 CP units 200 that make up the entire VLP.

### Discussion

Porcine circovirus 2 genome encodes for four known proteins: a replicase (ORF1) responsible for genome replication, a capsid protein (ORF2) responsible for generating the capsid shell, and an ORF3 and ORF4 that are believed to play a role in regulating cellular apoptosis (28, 29). Phylogenetic analysis of the PCV2 CP sequence indicates that here are 4 genotypes distributed globally (PCV2a-d) (14). PCV2b is currently believed to be the dominant genotype; however, the recent increase in the number of PCV2d CP depositions in GenBank suggests that there may be a shift in the genotype from PCV2b to PCV2d (5). The increase in the number of PCV2d entries may be a result of escape mutants in response to vaccination (17). To address the potential shift to the PCV2d genotype and the possibility that this new genotype may be resistant to the vaccines present on the market, the present application establishes a mammalian expression system for producing large quantities of PCV2d virus-like particles (VLP). This application provides the first system where mammalian cells have been utilized to generate a large quantity of VLP. The mammalian expression system is particularly advantageous to E. coli or baculovirus expression because it is more similar to cells naturally infected by PCV2, and thus allows for studying the details of the PCV2 capsid in the context of the viral replication cycle. For example, the N-terminus of the CP possesses multiple nuclear localization signals, and it is anticipated that virus assembly occurs in the nucleus of the infected cell where the ssDNA genome is replicated (30). However, it was previously unknown if PCV2 capsids can exit from intact nuclei to egress from the infected cells.

Consequently, in one or more embodiments, the present expression system can help address if the assembled capsid is capable of exiting from the nucleus. In one or more embodiments, the predominant fraction of VLP in the present expression system is located in the nucleus of the cell. This is unexpected because bioinformatic searches do not identify nuclear export signals (NES) in the CP sequence (data not shown). Thus, the PCV2 capsid may possess a cryptic NES that may play an important role in the viral life cycle. Nuclear cytoplasmic trafficking, however, can be reduced by redirecting capsid protein translation toward the secretory pathway by introducing a secretion signal sequence at the NH2 terminal of the capsid protein. This genetic modification results in an increase of capsid protein synthesis, VLP assembly and the release of the particles into the culture medium.

The cryo-EM image reconstruction and its structural analysis show that the mammalian, baculovirus and E. coli expressed VLP are nearly indistinguishable. Comparison of the PCV2a, b and d atomic coordinates identifies differences in the conformations of the surface-exposed loops consisting of amino acids 86-91 (loop CD), 131-136 (loop EF), and 188-194 (loop GH) (FIGS. 2A-2C). The structures of PCV2b and PCV2d are more similar to one another than either is with PCV2a. These differences are attributed to evolutionary coupled mutations in these loops (FIG. 3B) and provide a structural description for the antigenic shift observed in 2003 from PCV2a to PCV2b (15, 16). Symmetry expansion followed by focused classification of a capsid subunit do not identify any classes that may indicate post translational modification to the capsid (data not shown). Although, it may be necessary to use mass spectroscopy to further address the possibility of post-translational modification to PCV2 VLP generated from a mammalian expression system. Alignment of 1,377 unique PCV2 CP sequences indicates that only two amino acids (Met1 and Arg147) are absolutely conserved, and that the remaining positions in the sequence have undergone mutations. This demonstrates the remarkable plasticity of the capsid structure to undergo mutation while maintaining an infectious virus, and thus highlights the capacity of PCV2 to escape antibody neutralization. The atomic coordinates attained from the cryo-EM image reconstruction identify sites on the capsid surface that may be potentially targets for antibody neutralization. In the present application, continuous regions on the surface of the capsid are identified that may serve as epitopes that could potentially elicit neutralizing antibodies against several genotypes. The peptides that exhibit sequence variation may represent escape mutants from antibody neutralization. Consequently, the described expression protocol can be used to generate capsids assembled as a multivalent mosaic that simultaneously displays neutralizing epitopes of several genotypes. Positions that have not demonstrated variability could represent regions on the viral capsid that may be ideal for universal vaccine design.

The response to mammalian assembled VLP could more closely mimic an immune response to an actual infection. The mammalian VLP expression system could provide a recombinant vaccine of significant effectiveness. For instance, the antibody response of pigs inoculated with recombinant Cap protein derived from baculovirus to those that experienced a natural infection has been found to differ (31, 32). The recombinant vaccinated pigs preferentially recognized only the largest polypeptide fragment, CP (43-233) while experimentally infected pigs and pigs with PDNS showed strong reactivity against a CP oligopeptide, 169-180. The smaller peptide is common to PCV2a and PCV2b subtypes and could serve as a decoy that diverted the protective response from the larger 43-233 peptide.

Currently used vaccines have been produced using the capsid from a PCV2a genome and several studies have reported immunization failures as a consequence of PCV2d infection (7, 33, 34). In addition, there is always the possibility of low vaccine efficacy due to genomic shift and this factor must be accounted for in future vaccine development. G. Franzo et al (27) studied the vaccine-derived selection pressure cause by vaccination. They reported the high mutation rates at amino acid positions 59, 191, 206 for PCV2a and 131, 228 for PCV2b reduced the binding of antibody, that previously bound to the capsid; possibly causing the immune escape from vaccine protection (FIG. 4) (35). Consequently, a platform for expression of PCV2d VLP is warranted. As such, in accordance with at least one embodiment, the present application discloses an expression for producing PVC2d VLPs in mammalian cells.

In one or more embodiments, the VLP expression system of the present application has translational applications as well. The N-terminus 40 amino acids of CP are arginine rich, highly positively charged and presumed to interact with the ssDNA genome during viral morphogenesis. This motif locates in the interior space of the VLP and seems available for binding specific tags (or simply absorbing negatively charge small molecules or oligonucleotides) allowing the particle to be utilized as a nanoparticle for carrying therapeutic or immunodulatory moieties directed to specific tissues. Alternatively, the PCV2 N-terminus could be altered to hydrophobic amino acids for creating an internal hydrophobic environment for hydrophobic molecules. Such nanostructures could be utilized as diagnostic antigen or in a vaccine formulation. Furthermore, replacement of surface exposed amino acids with thiol containing residues or unnatural amino acids may allow for the conjugation of small, medium or large molecules useful for heterologous vaccination, drug delivery, therapeutic treatment, diagnostic, etc.

In accordance with one or more embodiments, an expression system of the present application can produce large quantities of the PVC2d VLPs in mammalian cells (human embryonic kidney: HEK 293). This system allows for rapid study of the PCV2 life-cycle in a background that closely mimics the natural host of PCV2. As explained in further detail below, the cryo-EM image reconstruction of the VLPs was determined to a resolution of 3.3 Å and used to identify potential antigenic epitopes that could be of use in a vaccine design or small therapeutic molecule delivery formulations. Comparison of 1,377 unique PCV2 CP entries in the GenBank indicates that except for the two amino acids every amino acid position has experienced a mutation. However, two groups of amino acids that form distinct patches on the surface of the capsid exhibit limited sequence variation. Vaccines capable of directing antibodies to these patches may serve as universal vaccines for PCV2.

### 6.2. Example 2

Vaccination-Challenge Study to Assess the Efficacy of Porcine Circovirus 2 Vaccines.

EXPERIMENTAL DESIGN. At approximately 1 week of age, blood samples from 120 male and female piglets were obtained at the source farm. These blood samples were analyzed for presence of PCV-2 by quantitative real time polymerase chain reaction (qRT-PCR) assay to discard any positive piglets. Those qRT-PCR PCV-2 negative samples, were tested for antibodies against PCV-2 using a commercial enzyme-linked immunosorbent assay (ELISA). Then, 72 two-week old male and female piglets that result to be negative to PCV-2 by qRT-PCR (coming from a litter with all the piglets tested negative) and with lowest levels of PCV-2 antibodies were selected and transported to the Test Site experimental farm. Upon arrival at the facilities, animals were weighed and the following day randomly distributed into six treatment groups of twelve pigs each, based on PCV-2 S/P titers (at screening), body weight, gender and sow parity. Subsequently, pigs were housed in four rooms for an acclimatization period of 10 days.

At 21+/−3 days of age (Study Day 0) pigs were immunized by intramuscular (IM) route on the right side of the neck. Piglets from treatment group T01 were immunized with 1 mL of vaccine PIGONE (Porcine circovirus type 2 (PCV2), strain VQ2610 1×10⁹-5×10⁹ copies of DNA equivalent to virus pre-activation). Piglets from treatment group T02 were immunized with 2 mL of vaccine PIGONE (Porcine circovirus type 2 (PCV2), strain VQ2610 1×10⁹-5×10⁹ copies of DNA equivalent to virus pre-activation) (hereinafter, “PIGONE”). Animals from T03 were immunized with 2 mL of vaccine Huve-PCV2 (6 with Huve-PCV2_A and 6 with Huve-PCV2_B). Huve-PCV2_A comprises a eukaryotic cell expressed PCV2d ORf2 protein and Huve-PCV2_B comprises a mammalian cell expressed PCV2d VLP. Animals from treatment group T04 were immunized with 1 mL of vaccine HVP-DNA (HVP-DNA comprises PCV2b ORF2 DNA vaccine) and animals from treatment T05 were immunized with 1 mL of the commercial vaccine Ingelvac CIRCOFLEX (Inactivated baculo-expressed PCV2 ORf2) (Boehringer Ingelheim). The piglets from treatment group T06 were administered phosphate-buffered saline (PBS) and used as a negative control group. On Study Day 21 (SD21), at approximately 6 weeks of age, pigs were immunized by intramuscular (IM) route on the left side of the neck. Pigs from treatment group T01 were immunized with 1 mL of vaccine PIGONE, pigs from treatment group T02 were immunized with 2 mL of vaccine PIGONE, animals from treatment group T03 were immunized with 2 mL of vaccine Huve-PCV2 (6 with Huve-PCV2_A and 6 with Huve-PCV2_B) and pigs from treatment group T04 were immunized with 1 mL of vaccine HVP-DNA. Groups T05 and T06 were intramuscularly immunized with 1 mL of PBS. Pigs were clinically inspected before each vaccination.

At approximately 8 weeks of age (WOA), on Study day 35, all animals from the six treatment groups were intranasally (IN) challenged with 3 mL of inoculum 104.95 TCID50/mL of PCV-2b strain Sp-10-′7-54-13.

Blood samples were collected weekly throughout the Study period for serology (ELISA) and/or PCV-2 detection in sera (qRT-PCR). Besides, nasal and rectal swabs were obtained weekly after challenge for PCV-2 shedding profile determination (if required). Animals were scored for clinical signs weekly after challenge and body weights were recorded at first vaccination, challenge and necropsy.

At 12 WOA (SD63 and SD64) all pigs from the five treatment groups were euthanized. At necropsy, tonsils, tracheobronchial lymph nodes, mesenteric lymph nodes and superficial inguinal lymph nodes were collected for histopathological examinations and PCV-2 detection in tissues (if required). The experimental design is represented in Table 2.

TREATMENT DISTRIBUTION. Animals were distributed in 6 groups of 12 animals (T01, T02, T03, T04, T05 or T06) according to PCV-2 antibody titers at screening, weight at day of arrival, gender and sow parity. The variables were ranked, then sorted and used to assign pigs to the six treatment groups in blocks of 6. Animals from treatments T01, T02, T03, T04 and T05 were randomly co-mingled in 3 rooms (rooms 5, 6 and 7), placing four animals from each group per room. Treatment T06 was in a separate room (room 8).

Animals from group T03 (Huve-PCV2) were divided in 2 subgroups: T03_A (Huve-PCV2_A) and T03_B (Huve-PCV2_B). This was done randomly on SD0, picking two animals from T03 from each room for each subgroup.

The room entry order was established as follows: immunity phase: room 8-room 7-room 6-room 5; challenge phase: room 5-room 6-room 7-room 8.

The room housing the PBS group (T06) was the first room to be entered during the immunity phase, and thereby the first room to be mock-vaccinated and challenged. After challenge, the rooms with vaccinated animals were accessed before entering the room with the control PBS group (T06).

TABLE 2
Study design summary

| | | | | | | | | Challenge. | | | |
|---|---|---|---|---|---|---|---|---|---|---|---|
| | | 1st Vaccination. | | | 2nd Vaccination. | | | 9 WOA (SD35) | | | Necropsy. 12 |
| | #of | 3 WOA (SD0) | | | 6 WOA (SD21) | | | | Virus | | WOA (SD63, SD64) |
| Grp. | pigs | Item | Rte. | Vol. | Item | Rte. | Vol. | Strain | titer* | Vol. | Samples |
| T01 | 12 | Pig | IM | 1 mL | Pig | IM | 1 mL | Sp-10- | 10^{4.95} | 3 mL | tonsil, |
| | | One | | | One | | | 7-54-13 | | | tracheobronchial, |
| | | | | | | | | | | | mesenteric and |
| | | | | | | | | | | | superficial |
| | | | | | | | | | | | inguinal lymph |
| | | | | | | | | | | | nodes |
| T02 | 12 | Pig | IM | 2 mL | Pig | IM | 2 mL | Sp-10- | 10^{4.95} | 3 mL | tonsil, |
| | | One | | | One | | | 7-54-13 | | | tracheobronchial, |
| | | | | | | | | | | | mesenteric and |
| | | | | | | | | | | | superficial |
| | | | | | | | | | | | inguinal lymph |
| | | | | | | | | | | | nodes |
| T03_A | 6 | Huve | IM | 2 mL | Huve | IM | 2 mL | Sp-10- | 10^{4.95} | 3 mL | tonsil, |
| | | PCV2_A | | | PCV2_A | | | 7-54-13 | | | tracheobronchial, |
| | | | | | | | | | | | mesenteric and |
| | | | | | | | | | | | superficial |
| | | | | | | | | | | | inguinal lymph |
| | | | | | | | | | | | nodes |
| T03_B | 6 | Huve | IM | 2 mL | Huve | IM | 2 mL | Sp-10- | 10^{4.95} | 3 mL | tonsil, |
| | | PCV2_A | | | PCV2_A | | | 7-54-13 | | | tracheobronchial, |
| | | | | | | | | | | | mesenteric and |
| | | | | | | | | | | | superficial |
| | | | | | | | | | | | inguinal lymph |
| | | | | | | | | | | | nodes |
| T04 | 12 | HVP | IM | 1 mL | HVP | IM | 1 mL | Sp-10- | 10^{4.95} | 3 mL | tonsil, |
| | | DNA | | | DNA | | | 7-54-13 | | | tracheobronchial, |
| | | | | | | | | | | | mesenteric and |
| | | | | | | | | | | | superficial |
| | | | | | | | | | | | inguinal lymph |
| | | | | | | | | | | | nodes |
| T05 | 12 | BI | IM | 1 mL | PBS | IM | 1 mL | Sp-10- | 10^{4.95} | 3 mL | tonsil, |
| | | Circo | | | | | | 7-54-13 | | | tracheobronchial, |
| | | flex | | | | | | | | | mesenteric and |
| | | | | | | | | | | | superficial |
| | | | | | | | | | | | inguinal lymph |
| | | | | | | | | | | | nodes |
| T06 | 12 | PBS | IM | 1 mL | PBS | IM | 1 mL | Sp-10- | 10^{4.95} | 3 mL | tonsil, |
| | | | | | | | | 7-54-13 | | | tracheobronchial, |
| | | | | | | | | | | | mesenteric and |
| | | | | | | | | | | | superficial |
| | | | | | | | | | | | inguinal lymph |
| | | | | | | | | | | | nodes |

Grp. = Group; IM = intramuscular; Rte. = Route; *TCID₅₀/mL.

Preliminary Results.

Health Events. Animal #472 from group T01 (PIGONE 2 mL) was found dead on SD32. Necropsy was performed and a fibrinous pericarditis was described. The cause of the death was probably due to bacterial septicaemia and not related to the Test Item.

Individual Animal Clinical Observations. All pigs were evaluated for depression, body condition and respiratory distress on the vaccination days (SD0 and SD21) pre-vaccination. Additionally, animals were also scored for clinical signs weekly after challenge, on Study days 35 pre-challenge, 41, 49, 56 and 63. No clinical signs were recorded for the Study animals during the Study phase.

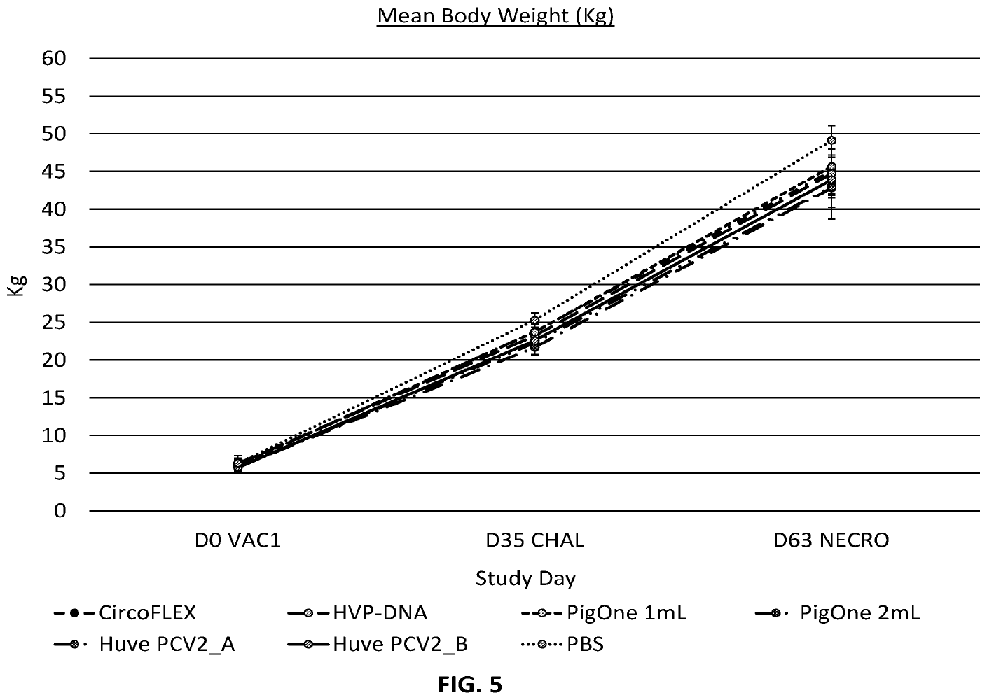

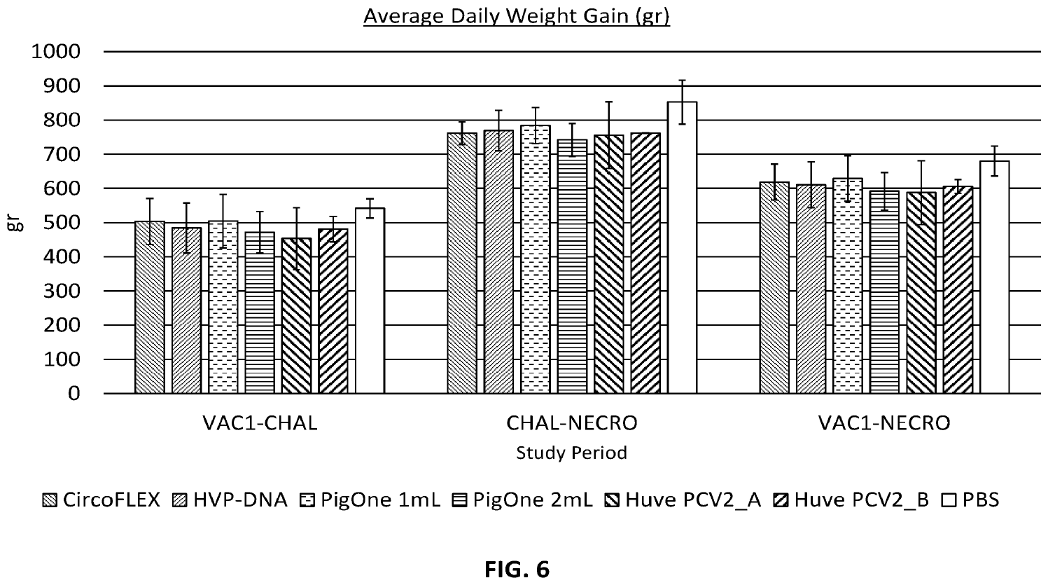

Body Weight. Body weights were recorded from all pigs at arrival at the experimental facility for treatment allocation purposes, at first vaccination (SD0), at challenge (SD35) and at necropsy (SD63). The average daily weight gain (ADWG) was calculated for the periods: first vaccination—challenge, challenge—necropsy and first vaccination—necropsy. Body weight evolution is observed in FIG. 5 and ADWG in FIG. 6.

PBS group (T06) had a slightly higher mean body weight on SD35 and SD63, and a higher ADWG on the three periods observed when compared to the other groups, but probably this is not statistically different.

Serology. Presence of antibodies against PCV-2 in blood in Study days SD0 pre-vaccination, SD7±1, SD13, SD20, SD29, SD35 pre-challenge, SD41, SD49, SD56 and SD63, were tested with the commercial ELISA IngezimCirco IgG kit. Results were expressed as S/P titers.

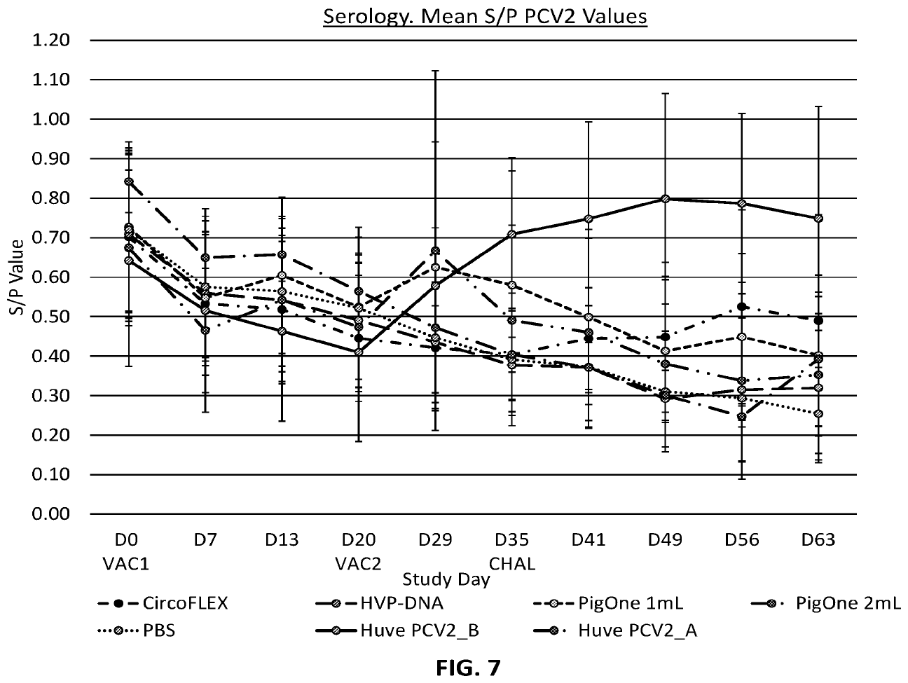

Antibody kinetics are represented in FIG. 7. Seven treatment groups are represented, treatment T03 (Huve-PCV2) is separated in two subgroups: Huve-PCV2_A (T03_A) and Huve-PCV2_B (T03_B).

At SD0 (first vaccination), S/P values of all Study animals ranged between 0.2 and 1, with a global mean value of 0.71.

A boost effect of the second vaccination is observed in 3 groups on SD29: PIGONE 1 mL and 2 mL (T01 and T02), and Huve-PCV2_B (T03).

At challenge day, groups with CIRCOFLEX (Inactivated baculo-expressed PCV2 ORf2) (T05), PBS (T06), Huve-PCV2_A (T03_A) and HVP-DNA (T04) had low S/P values, between 0.38-0.4. After challenge, generally S/P values decreased, and a mild response was observed for some vaccinated groups on SD56 or SD63.

The only group with high antibody response after second vaccination onwards was Huve-PCV2_B (T03_B).

Viremia. The commercial qRT-PCR kit LSI VetMAX Porcine Circovirus Type 2—Quantification (Life Technologies, reference code QPCV) was used to detect and quantify PCV-2 DNA in serum samples obtained on Study days SD0 pre-vaccination, SD35 prechallenge, SD41, SD49, SD56 and SD63. Results were expressed as PCV-2 copy numbers log₁₀/mL and classified as negative (<3 log₁₀ copies/mL), non-quantifiable positive (between 3 log₁₀ copies/mL and 4 log 10 copies/mL) or positive (≥4 log₁₀ copies/mL).

VIREMIA RESULTS CONSIDERING VIRAL LOADS >3 log₁₀ copies/mL.

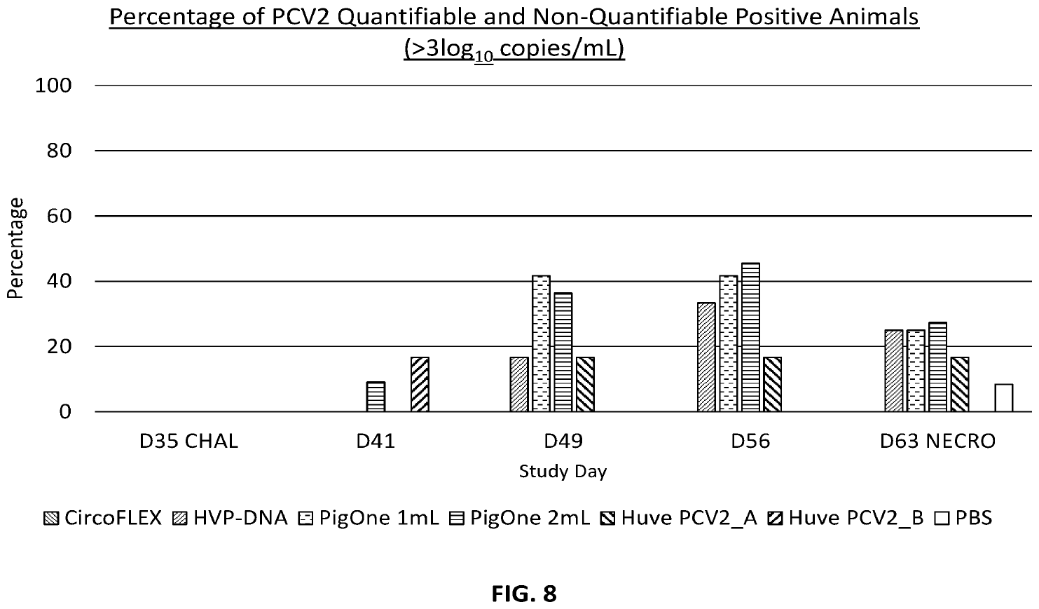

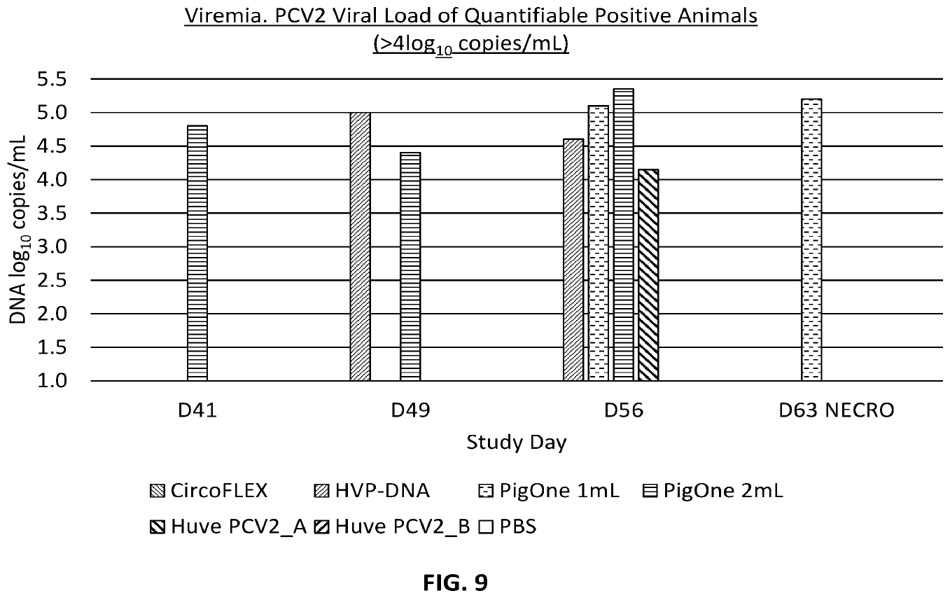

Results are represented in FIGS. 8 and 9, as percentage of quantifiable positive and non-quantifiable positive animals and mean values of quantifiable positive animals (>4 log₁₀ copies/mL).

Generally, a low percentage of positive animals (>3 log₁₀ copies/mL) is observed during the challenge phase. Treatment T01 and T02, vaccinated with PIGONE (1 mL or 2 mL) show the numerically higher percentages of positive animals on SD49 and SD56, but in any case, reaches 50% of positives (quantifiable+non-quantifiable). CIRCOFLEX (Inactivated baculo-expressed PCV2 ORf2) (T04) has no positive (quantifiable or non-quantifiable) animal during the challenge phase.

Huve-PCV2_B (T03_B), CIRCOFLEX (Inactivated baculo-expressed PCV2 ORf2) (T05) and PBS (T06) do not have quantifiable positive animals (>4 log₁₀ copies/mL) during the challenge phase. The remaining groups had between 1 and 3 positive animals on at least one sampling day during the challenge phase, being SD56 the day with more groups with positive animals. Mean values of quantifiable positive animals ranged between 4.12 log₁₀ and 5.34 log₁₀ PCV-2 copies/mL.

VIREMIA RESULTS CONSIDERING VIRAL LOADS >2 log₁₀ copies/mL.

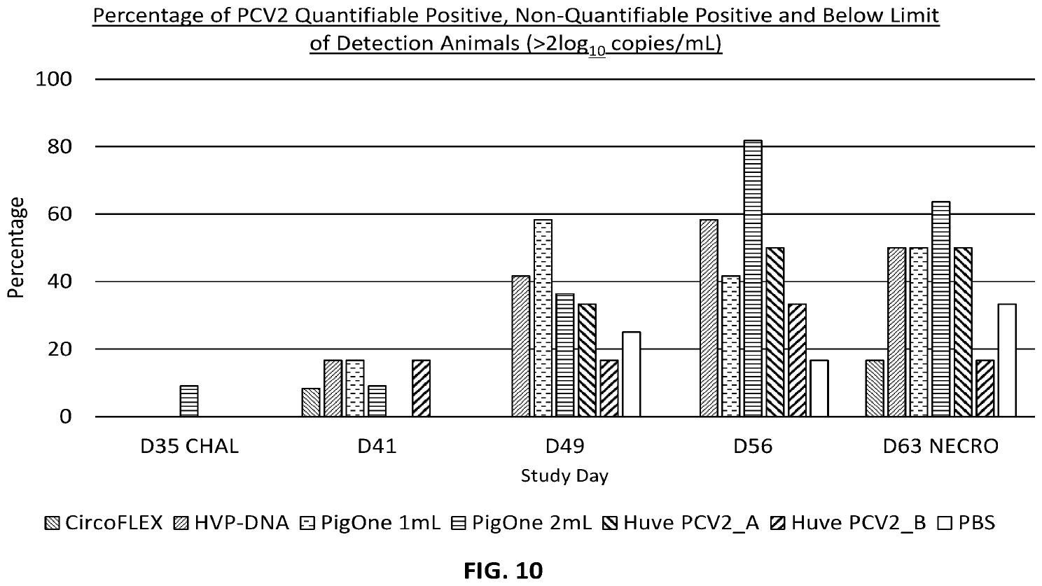

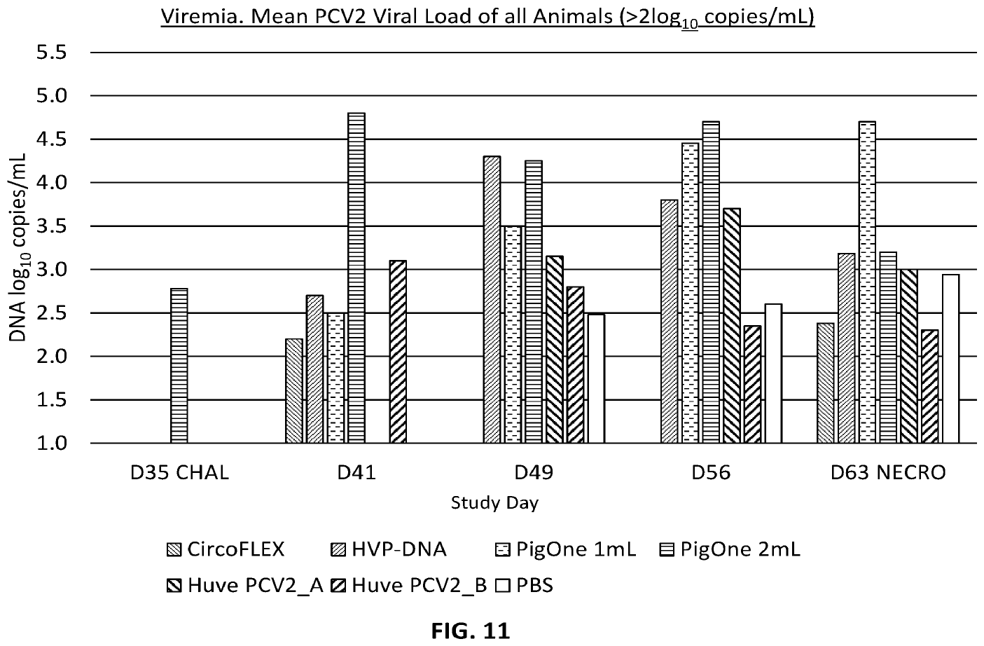

Results are represented in FIGS. 10 and 11, as percentage of viremic animals and mean values of all animals (>2 log₁₀ copies/mL).

When considering all animals, including below limit of detection samples, higher percentage of positives are observed in all Study groups. PIGONE groups (T01 or T02) continue to be the groups with numerically higher percentage of positives on SD49 (PIGONE 1 mL, T01), SD56 and SD63 (PIGONE 2 mL, T02), followed by HVPDNA (T04).

Mean viral loads considering all animals with qRT-PCR values ranged between 2.19-4.83 log₁₀ copies/mL.

Groups T01 (PIGONE 1 mL), T02 (PIGONE 2 mL), T03_B (Huve-PCV2_B) and T04 (HVP-DNA) present mean values >2 log₁₀ copies/mL during the four sampling days post-challenge. Huve-PCV2_A group (T03_A) and the PBS control group (T06) had positive values three days, from SD49 to SD63, while CIRCOFLEX (Inactivated baculo-expressed PCV2 ORf2) (T05) had viremic animals two days, at one week post challenge (SD41) and at necropsy (SD63).

At SD35, one animal from group T02 (PIGONE 2 mL) had a viral load of 2.76 log₁₀ copies/mL. This animal was negative for qRT-PCR on animal selection, SD0, SD7, SD14 and SD21. Also after challenge, on SD41 and SD49. During challenge, its viral load on SD56 and SD63 was >3 log₁₀ copies/mL.

Serology. S/P values at SD0 were higher than in the previous study due to maternal antibodies. This does not allow to see a clear seroconversion after vaccination. PIGONE 1 mL and 2 mL (TO1, T02) and Huve-PCV2_B (T03_B) apparently show seroconversion after 2nd vaccination when compared to PBS (T06). CIRCOFLEX (Inactivated baculo-expressed PCV2 ORf2) (T05) and HVP-DNA vaccine (T04) do not show antibody response. They have a similar antibody kinetics to that from the PBS group.

The lack of seroconversion on the CIRCOFLEX (Inactivated baculo-expressed PCV2 ORf2) group (T05) was expected taking into account the serological values at the time of vaccination. CIRCOFLEX (Inactivated baculo-expressed PCV2 ORf2) protection is based on cellular immunity. Normally, on field situation, no serological response is observed in animals with maternal antibodies when vaccinated with CIRCOFLEX (Inactivated baculo-expressed PCV2 ORf2).

Huve-PCV2 subgroups cannot be compared to the other groups as the n is half of the others (6 animals/subgroup). However, comparing among them, they have different results, as T03_B has a higher serological response.

Viremia. On SD35 before challenge, one animal from group PIGONE 2 mL (T02) was viremic, below limit of detection (viral load: 2.76 log₁₀ copies/mL). This animal was negative for qRT-PCR on animal selection, SD0, SD7, SD14 and SD21. Also after challenge, on SD41 and SD49. An attempt was made to try to sequence this DNA on SD35, but it was not possible due to the low viral load. Two possible explanations for this are: 1) subclinical infection with a field PCV-2 strain during the immunity phase with very low viral loads; and 2) a PCV-2 DNA contamination is being detected, not the entire virus. Unknown origin.

Generally, PCV-2 inoculation induced a mild subclinical infection as low viral loads were observed during challenge. Thereby, it was determined that all qRTPCR results, including ‘below limit of detection’ (2-3 log₁₀ copies/mL) should be considered. PIGONE 1 mL (T01), PIGONE 2 mL (T02) and HVP-DNA (T04) groups show a higher percentage of positive animals. Huve-PCV2_B (T03_B), CIRCOFLEX (Inactivated baculo-expressed PCV2 ORf2) (T05) and PBS (T06) groups have absence/very low percentage of viremic animals throughout challenge phase. Mean values are <3 log₁₀ copies/mL. When comparing Huve-PCV2 (T03) subgroups among them, subgroup T03_B has a numerically lower percentage of viremic animals and mean viral loads from SD49 to SD63.

Challenge Day Information. The inoculum was prepared as follows: it was thawed, mixed and then divided in 5 vials with approx. 50 mL/vial. Titration on challenge day was 104.95 TCID50. Vials were shared between rooms, i.e., one vial was started and until it was not finished a new one was not used, so one same vial could be used in two rooms. Inoculum vials were under refrigeration until use.

Challenge order: PBS group (T06) was challenged first (room 8) and then the vaccinated groups (rooms 7, 6, and 5). Possible room effect: PBS (T06) room had a lower density of animals than the other three rooms with the vaccinated groups. 12 or 20 animals/room, respectively. The 4 rooms were equal by means of space and housing conditions (temperature, humidity, etc.).

### FURTHER EMBODIMENTS

In accordance with one or more embodiments, the present application discloses an assembly of recombinant porcine circovirus capsids or virus-like particles (VLPs) comprising unique structural and antigens properties wherein one or more constituent capsid monomers incorporate sequences conferring monovalent or multivalent vaccine attributes and or the ability to encapsulate or mount molecular entities with distinct biological activities. In at least one embodiment, the recombinant capsid or VLP includes a secretion signal sequence genetically linked to the capsid coding sequence that allows for the release and or secretion of the particles from the producing cells into the culture medium.

In one or more embodiments, the recombinant capsid or VLP the sequence encoding surface motifs and loops are mutated, changed or modified hence the antigenic properties of the assembled VLP are new, different or chimeric.

In one or more embodiments of the recombinant capsid of VLP, the assembly and or production is carried out with two or more distinct capsid protein monomers resulting in VLPs of mixed composition (mosaic or chimeric) with unique antigenic and biological properties. In one or more embodiments, the recombinant capsid or VLP includes a modified surface that allows for the conjugation of different molecular entities to the surface of the VLP.

In one or more embodiments, the recombinant capsid or VLP includes an NH2 terminal or internal amino acids of the capsid monomers that are mutated, deleted and or modified allowing for the incorporation of small molecules, nucleic acids or other molecular moieties during assembly within the VLP producing cells or in vitro via dissemble and reassemble of the VLPs.

In one or more embodiments, the resulting VLPs target specific tissues or cells to delivery molecular entities and exert a biological activity.

In at least one embodiment, the present application also discloses a DNA construct comprising sequences encoding the capsid protein in all its sequence and form used to assemble the VLP. The DNA construct comprising sequences encoding the various form of the capsid monomers. In one or more embodiments, the present application further discloses a method of producing a VLP, the method comprising introducing into a host cell one or more of the DNA constructs under conditions such that the cell produces the VLP. In at least one embodiment, the host cell is a eukaryotic cell selected from the group consisting of mammalian, yeast, insect, plant, amphibian and avian cells. In one or more embodiments, the DNA construct is introduced into the cells and integrates within the cell genome or evenly divides and segregate into daughter cells, which results in the continuous production of VLPs.

In at least one embodiment, the present application discloses a VLP generated by the above method.

In one or more embodiments, an immunogenic composition is provided that comprises at least one VLP according to any of the above embodiments. In at least one embodiment, the immunogenic composition further comprises an adjuvant. In one or more embodiments, a method of generating an immune response to one or more porcine circovirus in an animal is provided. The method comprises administering to the animal an effective amount of the immunogenic composition. In at least one embodiment of the method, the composition is administered mucosally, intradermally, subcutaneously, intramuscularly, or orally. In at least one embodiment, the immune response vaccinates the animal against multiple serotypes or antigenic variants of one or more porcine circovirus. In at least one embodiment of the method, the animal is a swine.

In one or more embodiments, the recombinant capsid or VLP of the present application comprises molecules with pharmaceutical or biological activities. In at least one embodiment, the recombinant capsid or VLP comprises a pharmaceutical composition for VLP drug delivery, heterologous immunization, and or immunomodulation.

In one or more embodiments of the method for generating an immune response to one or more porcine circovirus in an animal, the immunogenic composition is administered to a subject as therapeutics or prophylactic treatment. The present methods provide for an effective use of the compositions of the present application in a subject. In at least one embodiment, the subject is a human or an animal.

### List of Sequences

SEQ ID NO: 1. Nucleotide sequence of wild type PCV2 capsid.
SEQ ID NO: 2. Amino acids sequence wild type PCV2 capsid.
SEQ ID NO: 3. Nucleotide sequence of modified PCV2 capsid with a secretion signal sequence.
SEQ ID NO: 4. Amino acids sequence of modified PCV2 capsid with secretion signal sequence.
SEQ ID NO: 5. Nucleotide sequence of Pcdna3.4-PCV2. (FIG. 1A).

Porcine circovirus-2 strain PCV2 capsid
Length: 705
Type: DNA
SEQ ID NO: 1
ATGACGTATC CAAGGAGGCG TTTCCGCAGA CGAAGACACC GCCCCCGCAG
CCATCTTGGC CAGATCCTCC GCCGCCGCCC CTGGCTCGTC CACCCCCGCC
ACCGTTACCG CTGGAGAAGG AAAAATGGCA TCTTCAACAC CCGCCTCTCC
CGCACCATCG GTTATACTGT CAAGAAAACC ACAGTCAGAA CGCCCTCCTG
GAATGTGGAC ATGATGAGAT TTAATATTAA TGATTTTCTT CCCCCAGGAG
GGGGCTCAAA CCCCCTCACT GTGCCCTTTG AATACTACAG AATAAGGAAG
GTTAAGGTTG AATTCTGGCC CTGCTCCCCA ATCACCCAGG GTGACAGGGG
AGTGGGCTCC ACTGCTGTTA TTCTAGATGA TAACTTTGTA ACAAAGGCCA
ATGCCCTAAC CTATGACCCC TATGTAAACT ACTCCTCCCG CCATACCATA
ACCCAGCCCT TCTCCTACCA CTCCCGGTAC TTTACCCCGA AACCTGTCCT
TGATAGGACA ATCGATTACT TCCAACCCAA TAACAAAAGA AATCAACTCT
GGCTGAGACT ACAAACTACT GGAAATGTAG ACCATGTAGG CCTCGGCACT
GCGTTCGAAA ACAGTATCTA CGACCAGGAC TACAATATCC GTATAACCAT
GTATGTACAA TTCAGAGAAT TTAATCTTAA AGACCCCCCA CTTAACCCTA AGTGA.
Porcine circovirus-2 strain PCV2 capsid
Length: 234
Type: Amino acid
SEQ ID NO: 2
M T Y P R R R F R R R R H R P R S H L G Q I L R R R P W L V H P R
H R Y R W R R K N G I F N T R L S R T I G Y T V K K T T V R T P S
W N V D M M R F N I N D F L P P G G G S N P L T V P F E Y Y R I R
K V K V E F W P C S P I T Q G D R G V G S T A V I L D D N F V T K
A N A L T Y D P Y V N Y S S R H T I T Q P F S Y H S R Y F T P K P
V L D R T I D Y F Q P N N K R N Q L W L R L Q T T G N V D H V G L
G T A F E N S I Y D Q D Y N I R I T M Y V Q F R E F N L K D P P L
N P K.
Modified Porcine circovirus-2 (PCV2) capsid
Length: 750
Type: DNA
SEQ ID NO: 3
ATGGAGAAAA TAGTGCTTCT TTTTGCAATA GTCAGTCTTG TTAAAAGTAC
GTATCCAAGG AGGCGTTTCC GCAGACGAAG ACACCGCCCC CGCAGCCATC
TTGGCCAGAT CCTCCGCCGC CGCCCCTGGC TCGTCCACCC CCGCCACCGT
TACCGCTGGA GAAGGAAAAA TGGCATCTTC AACACCCGCC TCTCCCGCAC
CATCGGTTAT ACTGTCAAGA AAACCACAGT CAGAACGCCC TCCTGGAATG
TGGACATGAT GAGATTTAAT ATTAATGATT TTCTTCCCCC AGGAGGGGGC
TCAAACCCCC TCACTGTGCC CTTTGAATAC TACAGAATAA GGAAGGTTAA
GGTTGAATTC TGGCCCTGCT CCCCAATCAC CCAGGGTGAC AGGGGAGTGG
GCTCCACTGC TGTTATTCTA GATGATAACT TTGTAACAAA GGCCAATGCC
CTAACCTATG ACCCCTATGT AAACTACTCC TCCCGCCATA CCATAACCCA
GCCCTTCTCC TACCACTCCC GGTACTTTAC CCCGAAACCT GTCCTTGATA
GGACAATCGA TTACTTCCAA CCCAATAACA AAAGAAATCA ACTCTGGCTG
AGACTACAAA CTACTGGAAA TGTAGACCAT GTAGGCCTCG GCACTGCGTT
CGAAAACAGT ATCTACGACC AGGACTACAA TATCCGTATA ACCATGTATG
TACAATTCAG AGAATTTAAT CTTAAAGACC CCCCACTTAA CCCTAAGTGA.
Modified porcine circovirus-2 strain PCV2 capsid
Length: 249
Type: Amino acid
SEQ ID NO: 4
M E K I V L L F A I V S L V K S T Y P R R R F R R R R H R P R S H
L G Q I L R R R P W L V H P R H R Y R W R R K N G I F N T R L S R
T I G Y T V K K T T V R T P S W N V D M M R F N I N D F L P P G G
G S N P L T V P F E Y Y R I R K V K V E F W P C S P I T Q G D R G
V G S T A V I L D D N F V T K A N A L T Y D P Y V N Y S S R H T I
T Q P F S Y H S R Y F T P K P V L D R T I D Y F Q P N N K R N Q L
W L R L Q T T G N V D H V G L G T A F E N S I Y D Q D Y N I R I T
M Y V Q F R E F N L K D P P L N P K.
Pcdna3.4-PCV2. (FIG. IA).
Length: 6778
Type: DNA
SEQ ID NO: 5
ATGACTTACC CTAGACGACG GTTCCGAAGA CGCAGACACA GACCGCGATC ACATCTCGGA
CAGATCCTTC GAAGAAGACC TTGGCTCGTT CATCCCCGGC ACAGATATAG ATGGCGAAGA
AAAAATGGAA TTTTCAATAC CCGCCTGAGC CGCACTATCG GGTACACCGT GAAAAAGACG
ACAGTGCGCA CCCCTTCCTG GAATGTCGAC ATGATGCGCT TCAACATAAA CGATTTCCTG
CCACCCGGAG GAGGAAGTAA TCCCCTGACT GTTCCTTTCG AATACTATAG AATAAGAAAA
GTGAAAGTGG AATTCTGGCC CTGCAGCCCC ATTACACAGG GAGACAGAGG TGTAGGCTCC
ACCGCTGTGA TTCTTGATGA CAATTTTGTG ACGAAAGCTA ATGCACTGAC CTACGACCCC
TACGTGAATT ACTCTAGCAG ACATACAATT ACCCAGCCCT TTTCCTACCA TTCCCGATAT
TTTACTCCAA AACCCGTCCT TGACAGAACT ATTGACTATT TTCAGCCCAA TAATAAACGC
AACCAACTCT GGCTCAGACT TCAGACTACT GGCAACGTGG ATCACGTCGG ACTTGGGACA
GCGTTCGAGA ACTCTATATA CGACCAAGAC TATAACATTC GCATTACAAT GTACGTGCAG
TTCAGAGAAT TCAATCTCAA AGACCCCCCA CTCAATCCAA AGTGAGCGGC CGCCCCGGGT
TCGAAACCGG TTAGTAATGA GTTTGATATC TCGACAATCA ACCTCTGGAT TACAAAATTT
GTGAAAGATT GACTGGTATT CTTAACTATG TTGCTCCTTT TACGCTATGT GGATACGCTG
CTTTAATGCC TTTGTATCAT GCTATTGCTT CCCGTATGGC TTTCATTTTC TCCTCCTTGT
ATAAATCCTG GTTGCTGTCT CTTTATGAGG AGTTGTGGCC CGTTGTCAGG CAACGTGGCG
TGGTGTGCAC TGTGTTTGCT GACGCAACCC CCACTGGTTG GGGCATTGCC ACCACCTGTC
AGCTCCTTTC CGGGACTTTC GCTTTCCCCC TCCCTATTGC CACGGCGGAA CTCATCGCCG
CCTGCCTTGC CCGCTGCTGG ACAGGGGCTC GGCTGTTGGG CACTGACAAT TCCGTGGTGT
TGTCGGGGAA GCTGACGTCC TTTCCATGGC TGCTCGCCTG TGTTGCCACC TGGATTCTGC
GCGGGACGTC CTTCTGCTAC GTCCCTTCGG CCCTCAATCC AGCGGACCTT CCTTCCCGCG
GCCTGCTGCC GGCTCTGCGG CCTCTTCCGC GTCTTCGCCT TCGCCCTCAG ACGAGTCGGA
TCTCCCTTTG GGCCGCCTCC CCGCCTGGAA ACGGGGGAGG CTAACTGAAA CACGGAAGGA
GACAATACCG GAAGGAACCC GCGCTATGAC GGCAATAAAA AGACAGAATA AAACGCACGG
GTGTTGGGTC GTTTGTTCAT AAACGCGGGG TTCGGTCCCA GGGCTGGCAC TCTGTCGATA
CCCCACCGAG ACCCCATTGG GGCCAATACG CCCGCGTTTC TTCCTTTTCC CCACCCCACC
CCCCAAGTTC GGGTGAAGGC CCAGGGCTCG CAGCCAACGT CGGGGCGGCA GGCCCTGCCA
TAGCAGATCT GCGCAGCTGG GGCTCTAGGG GGTATCCCCA CGCGCCCTGT AGCGGCGCAT
TAAGCGCGGC GGGTGTGGTG GTTACGCGCA GCGTGACCGC TACACTTGCC AGCGCCCTAG
CGCCCGCTCC TTTCGCTTTC TTCCCTTCCT TTCTCGCCAC GTTCGCCGGC TTTCCCCGTC
AAGCTCTAAA TCGGGGCATC CCTTTAGGGT TCCGATTTAG TGCTTTACGG CACCTCGACC
CCAAAAAACT TGATTAGGGT GATGGTTCAC GTAGTGGGCC ATCGCCCTGA TAGACGGTTT
TTCGCCCTTT GACGTTGGAG TCCACGTTCT TTAATAGTGG ACTCTTGTTC CAAACTGGAA
CAACACTCAA CCCTATCTCG GTCTATTCTT TTGATTTATA AGGGATTTTG GGGATTTCGG
CCTATTGGTT AAAAAATGAG CTGATTTAAC AAAAATTTAA CGCGAATTAA TTCTGTGGAA
TGTGTGTCAG TTAGGGTGTG GAAAGTCCCC AGGCTCCCCA GCAGGCAGAA GTATGCAAAG
CATGCATCTC AATTAGTCAG CAACCAGGTG TGGAAAGTCC CCAGGCTCCC CAGCAGGCAG
AAGTATGCAA AGCATGCATC TCAATTAGTC AGCAACCATA GTCCCGCCCC TAACTCCGCC
CATCCCGCCC CTAACTCCGC CCAGTTCCGC CCATTCTCCG CCCCATGGCT GACTAATTTT
TTTTATTTAT GCAGAGGCCG AGGCCGCCTC TGCCTCTGAG CTATTCCAGA AGTAGTGAGG
AGGCTTTTTT GGAGGCCTAG GCTTTTGCAA AAAGCTCCCG GGAGCTTGTA TATCCATTTT
CGGATCTGAT CAAGAGACAG GATGAGGATC GTTTCGCATG ATTGAACAAG ATGGATTGCA
CGCAGGTTCT CCGGCCGCTT GGGTGGAGAG GCTATTCGGC TATGACTGGG CACAACAGAC
AATCGGCTGC TCTGATGCCG CCGTGTTCCG GCTGTCAGCG CAGGGGCGCC CGGTTCTTTT
TGTCAAGACC GACCTGTCCG GTGCCCTGAA TGAACTGCAG GACGAGGCAG CGCGGCTATC
GTGGCTGGCC ACGACGGGCG TTCCTTGCGC AGCTGTGCTC GACGTTGTCA CTGAAGCGGG
AAGGGACTGG CTGCTATTGG GCGAAGTGCC GGGGCAGGAT CTCCTGTCAT CTCACCTTGC
TCCTGCCGAG AAAGTATCCA TCATGGCTGA TGCAATGCGG CGGCTGCATA CGCTTGATCC
GGCTACCTGC CCATTCGACC ACCAAGCGAA ACATCGCATC GAGCGAGCAC GTACTCGGAT
GGAAGCCGGT CTTGTCGATC AGGATGATCT GGACGAAGAG CATCAGGGGC TCGCGCCAGC
CGAACTGTTC GCCAGGCTCA AGGCGCGCAT GCCCGACGGC GAGGATCTCG TCGTGACCCA
TGGCGATGCC TGCTTGCCGA ATATCATGGT GGAAAATGGC CGCTTTTCTG GATTCATCGA
CTGTGGCCGG CTGGGTGTGG CGGACCGCTA TCAGGACATA GCGTTGGCTA CCCGTGATAT
TGCTGAAGAG CTTGGCGGCG AATGGGCTGA CCGCTTCCTC GTGCTTTACG GTATCGCCGC
TCCCGATTCG CAGCGCATCG CCTTCTATCG CCTTCTTGAC GAGTTCTTCT GAGCGGGACT
CTGGGGTTCG CGAAATGACC GACCAAGCGA CGCCCAACCT GCCATCACGA GATTTCGATT
CCACCGCCGC CTTCTATGAA AGGTTGGGCT TCGGAATCGT TTTCCGGGAC GCCGGCTGGA
TGATCCTCCA GCGCGGGGAT CTCATGCTGG AGTTCTTCGC CCACCCCAAC TTGTTTATTG
CAGCTTATAA TGGTTACAAA TAAAGCAATA GCATCACAAA TTTCACAAAT AAAGCATTTT
TTTCACTGCA TTCTAGTTGT GGTTTGTCCA AACTCATCAA TGTATCTTAT CATGTCTGTA
TACCGTCGAC CTCTAGCTAG AGCTTGGCGT AATCATGGTC ATAGCTGTTT CCTGTGTGAA
ATTGTTATCC GCTCACAATT CCACACAACA TACGAGCCGG AAGCATAAAG TGTAAAGCCT
GGGGTGCCTA ATGAGTGAGC TAACTCACAT TAATTGCGTT GCGCTCACTG CCCGCTTTCC
AGTCGGGAAA CCTGTCGTGC CAGCTGCATT AATGAATCGG CCAACGCGCG GGGAGAGGCG
GTTTGCGTAT TGGGCGCTCT TCCGCTTCCT CGCTCACTGA CTCGCTGCGC TCGGTCGTTC
GGCTGCGGCG AGCGGTATCA GCTCACTCAA AGGCGGTAAT ACGGTTATCC ACAGAATCAG
GGGATAACGC AGGAAAGAAC ATGTGAGCAA AAGGCCAGCA AAAGGCCAGG AACCGTAAAA
AGGCCGCGTT GCTGGCGTTT TTCCATAGGC TCCGCCCCCC TGACGAGCAT CACAAAAATC
GACGCTCAAG TCAGAGGTGG CGAAACCCGA CAGGACTATA AAGATACCAG GCGTTTCCCC
CTGGAAGCTC CCTCGTGCGC TCTCCTGTTC CGACCCTGCC GCTTACCGGA TACCTGTCCG
CCTTTCTCCC TTCGGGAAGC GTGGCGCTTT CTCAATGCTC ACGCTGTAGG TATCTCAGTT
CGGTGTAGGT CGTTCGCTCC AAGCTGGGCT GTGTGCACGA ACCCCCCGTT CAGCCCGACC
GCTGCGCCTT ATCCGGTAAC TATCGTCTTG AGTCCAACCC GGTAAGACAC GACTTATCGC
CACTGGCAGC AGCCACTGGT AACAGGATTA GCAGAGCGAG GTATGTAGGC GGTGCTACAG
AGTTCTTGAA GTGGTGGCCT AACTACGGCT ACACTAGAAG GACAGTATTT GGTATCTGCG
CTCTGCTGAA GCCAGTTACC TTCGGAAAAA GAGTTGGTAG CTCTTGATCC GGCAAACAAA
CCACCGCTGG TAGCGGTGGT TTTTTTGTTT GCAAGCAGCA GATTACGCGC AGAAAAAAAG
GATCTCAAGA AGATCCTTTG ATCTTTTCTA CGGGGTCTGA CGCTCAGTGG AACGAAAACT
CACGTTAAGG GATTTTGGTC ATGAGATTAT CAAAAAGGAT CTTCACCTAG ATCCTTTTAA
ATTAAAAATG AAGTTTTAAA TCAATCTAAA GTATATATGA GTAAACTTGG TCTGACAGTT
ACCAATGCTT AATCAGTGAG GCACCTATCT CAGCGATCTG TCTATTTCGT TCATCCATAG
TTGCCTGACT CCCCGTCGTG TAGATAACTA CGATACGGGA GGGCTTACCA TCTGGCCCCA
GTGCTGCAAT GATACCGCGA GACCCACGCT CACCGGCTCC AGATTTATCA GCAATAAACC
AGCCAGCCGG AAGGGCCGAG CGCAGAAGTG GTCCTGCAAC TTTATCCGCC TCCATCCAGT
CTATTAATTG TTGCCGGGAA GCTAGAGTAA GTAGTTCGCC AGTTAATAGT TTGCGCAACG
TTGTTGCCAT TGCTACAGGC ATCGTGGTGT CACGCTCGTC GTTTGGTATG GCTTCATTCA
GCTCCGGTTC CCAACGATCA AGGCGAGTTA CATGATCCCC CATGTTGTGC AAAAAAGCGG
TTAGCTCCTT CGGTCCTCCG ATCGTTGTCA GAAGTAAGTT GGCCGCAGTG TTATCACTCA
TGGTTATGGC AGCACTGCAT AATTCTCTTA CTGTCATGCC ATCCGTAAGA TGCTTTTCTG
TGACTGGTGA GTACTCAACC AAGTCATTCT GAGAATAGTG TATGCGGCGA CCGAGTTGCT
CTTGCCCGGC GTCAATACGG GATAATACCG CGCCACATAG CAGAACTTTA AAAGTGCTCA
TCATTGGAAA ACGTTCTTCG GGGCGAAAAC TCTCAAGGAT CTTACCGCTG TTGAGATCCA
GTTCGATGTA ACCCACTCGT GCACCCAACT GATCTTCAGC ATCTTTTACT TTCACCAGCG
TTTCTGGGTG AGCAAAAACA GGAAGGCAAA ATGCCGCAAA AAAGGGAATA AGGGCGACAC
GGAAATGTTG AATACTCATA CTCTTCCTTT TTCAATATTA TTGAAGCATT TATCAGGGTT
ATTGTCTCAT GAGCGGATAC ATATTTGAAT GTATTTAGAA AAATAAACAA ATAGGGGTTC
CGCGCACATT TCCCCGAAAA GTGCCACCTG ACGTCGACGG ATCGGGAGAT CTCCCGATCC
CCTATGGTCG ACTCTCAGTA CAATCTGCTC TGATGCCGCA TAGTTAAGCC AGTATCTGCT
CCCTGCTTGT GTGTTGGAGG TCGCTGAGTA GTGCGCGAGC AAAATTTAAG CTACAACAAG
GCAAGGCTTG ACCGACAATT GCATGAAGAA TCTGCTTAGG GTTAGGCGTT TTGCGCTGCT
TCGCGATGTA CGGGCCAGAT ATACGCGTTG ACATTGATTA TTGACTAGTT ATTAATAGTA
ATCAATTACG GGGTCATTAG TTCATAGCCC ATATATGGAG TTCCGCGTTA CATAACTTAC
GGTAAATGGC CCGCCTGGCT GACCGCCCAA CGACCCCCGC CCATTGACGT CAATAATGAC
GTATGTTCCC ATAGTAACGC CAATAGGGAC TTTCCATTGA CGTCAATGGG TGGAGTATTT
ACGGTAAACT GCCCACTTGG CAGTACATCA AGTGTATCAT ATGCCAAGTA CGCCCCCTAT
TGACGTCAAT GACGGTAAAT GGCCCGCCTG GCATTATGCC CAGTACATGA CCTTATGGGA
CTTTCCTACT TGGCAGTACA TCTACGTATT AGTCATCGCT ATTACCATGG TGATGCGGTT
TTGGCAGTAC ATCAATGGGC GTGGATAGCG GTTTGACTCA CGGGGATTTC CAAGTCTCCA
CCCCATTGAC GTCAATGGGA GTTTGTTTTG GCACCAAAAT CAACGGGACT TTCCAAAATG
TCGTAACAAC TCCGCCCCAT TGACGCAAAT GGGCGGTAGG CGTGTACGGT GGGAGGTCTA
TATAAGCAGA GCTCGTTTAG TGAACCGTCA GATCGCCTGG AGACGCCATC CACGCTGTTT
TGACCTCCAT AGAAGACACC GGGACCGATC CAGCCTCCGG ACTCTAGAGG ATCGAACCCT
TAAGCTTGGA TCCACTAGTG AATTCATCTA AGGTACCAGT CCAGCTAGCG CCGCCACC.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, and publications are cited throughout this application, the disclosures of which, particularly, including all disclosed chemical structures, are incorporated herein by reference. Citation of the above publications or documents is not intended as an admission that any of the foregoing is pertinent prior art, nor does it constitute any admission as to the contents or date of these publications or documents. All references cited herein are incorporated by reference to the same extent as if each individual publication, patent application, or patent, was specifically and individually indicated to be incorporated by reference.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

Exemplary systems and methods are set out in the following items:

Item 1: A mammalian expression system for producing recombinant porcine circovirus type 2 (PCV2) virus-like particles (VLPs), the expression system comprising:

a mammalian cell;

a plasmid comprising a PCV2 gene encoding a capsid protein, wherein the PCV2 gene is codon optimized;

wherein the mammalian cell is transfected with the plasmid; and

wherein the expression system produces recombinant PCV2 VLPs.

Item 2: The expression system of item 1, wherein the mammalian cell is a human embryonic kidney-293 (HEK-293) mammalian cell.

Item 3: The expression system of items 1-2, wherein the PCV2 gene includes a recognition site for NheI, a Kozak sequence, and a recognition site for NotI, and wherein the recognition site for NheI and the Kozak sequence are upstream from a start codon of the PCV2 gene, and the recognition site for NotI is incorporated after a termination codon of the PCV2 gene.

Item 4: The expression system of items 1-3, wherein a majority of the produced recombinant PCV2 VLPs are present in the nucleus of the mammalian cells.

Item 5: The expression system of items 1-4, wherein the capsid protein comprises an amino acid sequence of SEQ ID NO: 2.

Item 6: The expression system of items 1-5, wherein the capsid protein is encoded by a nucleotide sequence of SEQ ID NO: 1.

Item 7: The expression system of items 1-4, wherein the capsid protein is modified with a secretion signal sequence introduced at an NH2 terminal of the capsid protein.

Item 8: The expression system of item 7, wherein the capsid protein comprises an amino acid sequence of SEQ ID NO: 4.

Item 9: The expression system of items 7-8, wherein the capsid protein is encoded by a nucleotide sequence of SEQ ID NO: 3.

Item 10: The expression system of items 1-9, wherein the produced recombinant PCV2 VLPs are selected from the group consisting of: PCV2a VLPs, PCV2b VLPs, PCV2c VLPs, PCV2d VLPs, and PCV2e VLPs.

Item 11: The expression system of items 1-10, wherein the produced recombinant PCV2 VLPs are PVC2d VLPs.

Item 12: The expression system of items 1-11, wherein the plasmid is pcDNA3.4-PCV2.

Item 13: The expression system of items 1-12, wherein the PCV2 gene is codon optimized using a codon-optimized amino acid sequence of FIG. 1A.

Item 14: A method for producing porcine circovirus type 2 (PCV2) virus-like particles (VLPs), comprising:

providing a suspension of cultured mammalian cells;

transfecting the mammalian cells with a plasmid comprising a PCV2 gene encoding a capsid protein;

adding valproic acid (VPA) sodium salt to the transfected mammalian cells, wherein the addition of the VPA sodium salt inhibits cell proliferation;

centrifuging and washing the transfected mammalian cells;

suspending the centrifuged mammalian cells in a phosphate buffered saline (PBS) solution;

performing multiple freeze and thaw cycles on the mammalian cells;

sonicating the mammalian cells in multiple cycles; and

performing two successive centrifugation cycles of the mammalians cells to produce the PCV2 VLPs, wherein a majority of the produced PCV2 VLPs are present in the nucleus of the mammalian cells.

Item 15: The method of item 14, wherein the step of centrifuging and washing the transfected mammalian cells comprises:

centrifuging the mammalian cells at 2,000×g for 15 min;

washing the mammalian cells with PBS solution; and

centrifuging the mammalian cells again at 2,000×g for 15 min.

Item 16: The method of items 14-15, wherein the centrifuged mammalian cells are frozen at approximately −80° C. and thawed at approximately 37° C. during the freeze and thaw cycles.

Item 17: The method of items 14-16, wherein a first of the two successive centrifugation cycles is performed at 2,000×g for 15 min and a second of the two successive centrifugation cycles is performed at 8,000×g for 15 min.

Item 18: The method of items 14-17, further comprising purifying the PCV2 VLPs by ultracentrifugation.

Item 19: The method of items 14-18, wherein the mammalian cells are human embryonic kidney-293 (HEK-293) mammalian cells.

Item 20: The method of items 14-19, wherein the PCV2 gene includes a recognition site for NheI, a Kozak sequence, and a recognition site for NotI, and wherein the recognition site for NheI and the Kozak sequence are upstream from a start codon of the PCV2 gene, and the recognition site for NotI is incorporated after a termination codon of the PCV2 gene.

Item 21: The method of items 14-20, wherein the plasmid is pcDNA3.4-PCV2.

Items 22: The method of items 14-21, wherein the produced PCV2 VLPs are selected from the group consisting of: PCV2a VLPs, PCV2b VLPs, PCV2c VLPs, PCV2d VLPs, and PCV2e VLP.

Item 23: The method of items 14-22, wherein the produced PCV2 VLPs are PVC2d VLPs.

Item 24: A PCV2 VLP generated by the method of items 14-23.

### REFERENCES

1. Ellis J. 2014. Porcine circovirus: a historical perspective. Vet Pathol 51:315-27. 455
2. Rosario K, Breitbart M, Harrach B, Segales J, Delwart E, Biagini P, Varsani A. 2017. Revisiting the taxonomy of the family Circoviridae: establishment of the genus Cyclovirus and removal of the genus Gyrovirus. Arch Virol 162:1447-1463. 458
3. Breitbart M, Delwart E, Rosario K, Segales J, Varsani A, Ictv Report C. 2017. ICTV Virus Taxonomy Profile: Circoviridae. J Gen Virol 98:1997-1998.
4. Palinski R, Pineyro P, Shang P, Yuan F, Guo R, Fang Y, Byers E, Hause B M. 2017. A Novel Porcine Circovirus Distantly Related to Known Circoviruses Is Associated with Porcine Dermatitis 462 and Nephropathy Syndrome and Reproductive Failure. J Virol 91.
5. Wei C, Zhang M, Chen Y, Xie J, Huang Z, Zhu W, Xu T, Cao Z, Zhou P, Su S, Zhang G. 2013. Genetic evolution and phylogenetic analysis of porcine circovirus type 2 infections in southern China from 2011 to 2012. Infect Genet Evol 17:87-92.
6. Franzo G, Cortey M, Olvera A, Novosel D, Castro A M, Biagini P, Segales J, Drigo M. 2015. Revisiting the taxonomical classification of Porcine Circovirus type 2 (PCV2): still a real challenge. 468 Virol J 12:131.
7. Xiao C T, Halbur P G, Opriessnig T. 2015. Global molecular genetic analysis of porcine circovirus type 2 (PCV2) sequences confirms the presence of four main PCV2 genotypes and reveals a rapid increase of PCV2d. J Gen Virol 96:1830-41.
8. Wang N, Zhan Y, Wang A, Zhang L, Khayat R, Yang Y. 2016. In silico analysis of surface structure variation of PCV2 capsid resulting from loop mutations of its capsid protein (Cap). J Gen Virol 97:3331-3344.
9. Liu Z, Guo F, Wang F, Li T C, Jiang W. 2016. 2.9 A Resolution Cryo-EM 3D Reconstruction of Close-Packed Virus Particles. Structure 24:319-28.
10. Khayat R, Johnson J E. 2011. Pass the jelly rolls. Structure 19:904-6. 478
11. Khayat R, Brunn N, Speir J A, Hardham J M, Ankenbauer R G, Schneemann A, Johnson J E. 2011. The 2.3-angstrom structure of porcine circovirus 2. J Virol 85:7856-62.
12. Guo L, Lu Y, Huang L, Wei Y, Liu C. 2011. Identification of a new antigen epitope in the nuclear localization signal region of porcine circovirus type 2 capsid protein. Intervirology 54:156-63.
13. Mahe D, Blanchard P, Truong C, Arnauld C, Le Cann P, Cariolet R, Madec F, Albina E, Jestin A. 2000. Differential recognition of ORF2 protein from type 1 and type 2 porcine circoviruses and identification of immunorelevant epitopes. J Gen Virol 81:1815-24.
14. Guo L J, Lu Y H, Wei Y W, Huang L P, Liu C M. 2010. Porcine circovirus type 2 (PCV2): genetic variation and newly emerging genotypes in China. Virol J 7:273.
15. Beach N M, Meng X J. 2012. Efficacy and future prospects of commercially available and experimental vaccines against porcine circovirus type 2 (PCV2). Virus Res 164:33-42.
16. Patterson A R, Opriessnig T. 2010. Epidemiology and horizontal transmission of porcine circovirus type 2 (PCV2). Anim Health Res Rev 11:217-34.
17. Xiao C T, Halbur P G, Opriessnig T. 2012. Complete genome sequence of a novel porcine circovirus type 2b variant present in cases of vaccine failures in the United States. J Virol 86:12469.
18. Dhindwal S, Avila B, Feng S, Khayat R. 2019. Porcine circovirus 2 uses a multitude of weak binding sites to interact with heparan sulfate, and the interactions do not follow the symmetry of the capsid. Journal of Virology doi:10.1128/jvi.02222-18.
19. Sievers F, Wilm A, Dineen D, Gibson T J, Karplus K, Li W, Lopez R, McWilliam H, Remmert M, Soding J, Thompson J D, Higgins D G. 2011. Fast, scalable generation of high-quality protein multiple sequence alignments using Clustal Omega. Mol Syst Biol 7:539.
20. Ashkenazy H, Abadi S, Martz E, Chay O, Mayrose I, Pupko T, Ben-Tal N. 2016. ConSurf 2016: an improved methodology to estimate and visualize evolutionary conservation in macromolecules. Nucleic Acids Res 44: W344-50.
22. Hopf T A, Ingraham J B, Poelwijk F J, Scharfe C P I, Springer M, Sander C, Marks D S. 2017. Mutation effects predicted from sequence co-variation. Nature Biotechnology 35:128.
23. Shamsi Z, Moffett A S, Shukla D. 2017. Enhanced unbiased sampling of protein dynamics using evolutionary coupling information. Sci Rep 7:12700.
24. Fraczkiewicz R, Braun W. 1998. Exact and efficient analytical calculation of the accessible surface areas and their gradients for macromolecules. Journal of Computational Chemistry 19:319-333.
25. Saha D, Huang L, Bussalleu E, Lefebvre D J, Fort M, Van Doorsselaere J, Nauwynck H J. 2012. 513 Antigenic subtyping and epitopes' competition analysis of porcine circovirus type 2 using monoclonal antibodies. Vet Microbiol 157:13-22.
26. Lefebvre D J, Costers S, Van Doorsselaere J, Misinzo G, Delputte P L, Nauwynck H J. 2008. Antigenic differences among porcine circovirus type 2 strains, as demonstrated by the use of monoclonal antibodies. J Gen Virol 89:177-87.
27. Franzo G, Tucciarone C M, Cecchinato M, Drigo M. 2016. Porcine circovirus type 2 (PCV2) evolution before and after the vaccination introduction: A large scale epidemiological study. Sci Rep 6:39458.
28. Teras M, Viisileht E, Pahtma-Hall M, Rump A, Paalme V, Pata P, Pata I, Langevin C, Ruutel 522 Boudinot S. 2018. Porcine circovirus type 2 ORF3 protein induces apoptosis in melanoma cells. BMC Cancer 18:1237.
29. Pan Y, Li P, Jia R, Wang M, Yin Z, Cheng A. 2018. Regulation of Apoptosis During Porcine Circovirus Type 2 Infection. Front Microbiol 9:2086.
30. Shuai J, Wei W, Jiang L, Li Xl, Chen N, Fang W. 2008. Mapping of the nuclear localization signals in open reading frame 2 protein from porcine circovirus type 1. Acta Biochimica et Biophysica Sinica 40:71-77.
31. Trible B R, Suddith A W, Kerrigan M A, Cino-Ozuna A G, Hesse R A, Rowland R R. 2012. Recognition of the different structural forms of the capsid protein determines the outcome following infection with porcine circovirus type 2. J Virol 86:13508-14.
32. Trible B R, Kerrigan M, Crossland N, Potter M, Faaberg K, Hesse R, Rowland R R. 2011. Antibody recognition of porcine circovirus type 2 capsid protein epitopes after vaccination, infection, and disease. Clin Vaccine Immunol 18:749-57.
33. Seo H W, Park C, Kang I, Choi K, Jeong J, Park S J, Chae C. 2014. Genetic and antigenic characterization of a newly emerging porcine circovirus type 2b mutant first isolated in cases of vaccine failure in Korea. Arch Virol 159:3107-11.
34. Opriessnig T, Gerber P F, Xiao C T, Halbur P G, Matzinger S R, Meng X J. 2014. Commercial PCV2a-based vaccines are effective in protecting naturally PCV2b-infected finisher pigs against experimental challenge with a 2012 mutant PCV2. Vaccine 32:4342-8.
35. Franzo G, Cecchinato M, Martini M, Ceglie L, Gigli A, Drigo M. 2014. Observation of high recombination occurrence of Porcine Reproductive and Respiratory Syndrome Virus in field condition. Virus Res 194:159-66.
36. Suloway C, Shi J, Cheng A, Pulokas J, Carragher B, Potter C S, Zheng S Q, Agard D A, Jensen G J. 2009. Fully automated, sequential tilt-series acquisition with Leginon. J Struct Biol 167:11-8.
37. Zheng S Q, Palovcak E, Armache J P, Verba K A, Cheng Y, Agard D A. 2017. MotionCor2: anisotropic correction of beam-induced motion for improved cryo-electron microscopy. Nat Methods 14:331-332.
38. Zhang T, Gu Y, Fan H. 2016. Effect of impurities and post-experimental purification in SAD phasing with serial femtosecond crystallography data. Acta Crystallogr D Struct Biol 72:789-94.
39. Scheres S H. 2012. RELION: implementation of a Bayesian approach to cryo-EM structure determination. J Struct Biol 180:519-30.
40. Pettersen E F, Goddard T D, Huang C C, Couch G S, Greenblatt D M, Meng E C, Ferrin T E. 2004. UCSF Chimera—a visualization system for exploratory research and analysis. J Comput Chem 25:1605-12.
41. Vilas J L, Gomez-Blanco J, Conesa P, Melero R, Miguel de la Rosa-Trevin J, Oton J, Cuenca J, Marabini R, Carazo J M, Vargas J, Sorzano C O S. 2018. MonoRes: Automatic and Accurate Estimation of Local Resolution for Electron Microscopy Maps. Structure 26:337-344 e4.
42. Emsley P, Cowtan K. 2004. Coot: model-building tools for molecular graphics. Acta Crystallogr D Biol Crystallogr 60:2126-32.
43. Afonine P A, Headd J J, Terwilliger T C, Adams P C. 2013. Computational Crystallography Newsletter 4:43-44.
44. Johnson M, Zaretskaya I, Raytselis Y, Merezhuk Y, McGinnis S, Madden T L. 2008. NCBI

BLAST: a better web interface. Nucleic Acids Res 36: W5-9.
45. Edgar R C. 2004. MUSCLE: multiple sequence alignment with high accuracy and high throughput. Nucleic Acids Res 32:1792-7.
46. Martin D P, Lemey P, Lott M, Moulton V, Posada D, Lefeuvre P. 2010. RDP3: a flexible and fast computer program for analyzing recombination. Bioinformatics 26:2462-3.
47. Goddard T D, Huang C C, Meng E C, Pettersen E F, Couch G S, Morris J H, Ferrin T E. 2018. UCSF ChimeraX: Meeting modern challenges in visualization and analysis. Protein Sci 27:14-25.

1. A mammalian expression system for producing recombinant porcine circovirus type 2 (PCV2) virus-like particles (VLPs), the expression system comprising:
a mammalian cell;
a plasmid comprising a PCV2 gene encoding a capsid protein, wherein the PCV2 gene is codon optimized;
wherein the mammalian cell is transfected with the plasmid; and
wherein the expression system produces recombinant PCV2 VLPs.

2. The expression system of claim 1, wherein the mammalian cell is a human embryonic kidney-293 (HEK-293) mammalian cell.

3. The expression system of claim 1, wherein the PCV2 gene includes a recognition site for NheI, a Kozak sequence, and a recognition site for NotI, and
wherein the recognition site for NheI and the Kozak sequence are upstream from a start codon of the PCV2 gene, and the recognition site for NotI is incorporated after a termination codon of the PCV2 gene.

4. The expression system of claim 1, wherein a majority of the produced recombinant PCV2 VLPs are present in the nucleus of the mammalian cells.

5. The expression system of claim 1, wherein the capsid protein comprises an amino acid sequence of SEQ ID NO: 2.

6. The expression system of claim 1, wherein the capsid protein is encoded by a nucleotide sequence of SEQ ID NO: 1.

7. The expression system of claim 1, wherein the capsid protein is modified with a secretion signal sequence introduced at an NH2 terminal of the capsid protein.

8. The expression system of claim 7, wherein the capsid protein comprises an amino acid sequence of SEQ ID NO: 4.

9. The expression system of claim 7, wherein the capsid protein is encoded by a nucleotide sequence of SEQ ID NO: 3.

10. The expression system of claim 1, wherein the produced recombinant PCV2 VLPs are selected from the group consisting of: PCV2a VLPs, PCV2b VLPs, PCV2c VLPs, PCV2d VLPs, and PCV2e VLPs.

11. (canceled)

12. The expression system of claim 1, wherein the plasmid is pcDNA3.4-PCV2.

13. (canceled)

14. A method for producing porcine circovirus type 2 (PCV2) virus-like particles (VLPs), comprising:
providing a suspension of cultured mammalian cells;
transfecting the mammalian cells with a plasmid comprising a PCV2 gene encoding a capsid protein;
adding valproic acid (VPA) sodium salt to the transfected mammalian cells, wherein the addition of the VPA sodium salt inhibits cell proliferation;
centrifuging and washing the transfected mammalian cells;
suspending the centrifuged mammalian cells in a phosphate buffered saline (PBS) solution;
performing multiple freeze and thaw cycles on the mammalian cells;
sonicating the mammalian cells in multiple cycles; and
performing two successive centrifugation cycles of the mammalians cells to produce the PCV2 VLPs,
wherein a majority of the produced PCV2 VLPs are present in the nucleus of the mammalian cells.

15. The method of claim 14, wherein the step of centrifuging and washing the transfected mammalian cells comprises:
centrifuging the mammalian cells at 2,000×g for 15 min;
washing the mammalian cells with PBS solution; and
centrifuging the mammalian cells again at 2,000×g for 15 min.

16. The method of claim 14, wherein the centrifuged mammalian cells are frozen at approximately −80° C. and thawed at approximately 37° C. during the freeze and thaw cycles.

17. The method of claim 14, wherein a first of the two successive centrifugation cycles is performed at 2,000×g for 15 min and a second of the two successive centrifugation cycles is performed at 8,000×g for 15 min.

18. The method of claim 14, further comprising purifying the PCV2 VLPs by ultracentrifugation.

19. The method of claim 14, wherein the mammalian cells are human embryonic kidney-293 (HEK-293) mammalian cells.

20. The method of claim 14, wherein the PCV2 gene includes a recognition site for NheI, a Kozak sequence, and a recognition site for NotI, and
wherein the recognition site for NheI and the Kozak sequence are upstream from a start codon of the PCV2 gene, and the recognition site for NotI is incorporated after a termination codon of the PCV2 gene.

21. The method of claim 14, wherein the plasmid is pcDNA3.4-PCV2.

22. The method of claim 14, wherein the produced PCV2 VLPs are selected from the group consisting of: PCV2a VLPs, PCV2b VLPs, PCV2c VLPs, PCV2d VLPs, and PCV2e VLP.

23. (canceled)

24. (canceled)
